(19) United States
(12) Patent Application Publication
(10) Pub. No.: US 2024/0136020 A1
(43) Pub. Date: Apr. 25, 2024

(54) RNA-PROTEIN INTERACTION PREDICTION METHOD AND APPARATUS, AND MEDIUM AND ELECTRONIC DEVICE

(71) Applicant: BOE Technology Group Co., Ltd., Beijing (CN)

(72) Inventor: Zhenzhong ZHANG, Beijing (CN)

(21) Appl. No.: 18/264,254

(22) PCT Filed: Nov. 30, 2021
(86) PCT No.: PCT/CN2021/134640

§ 371 Date: Aug. 4, 2023

(51) Int. Cl. G16B 40/00; G06N 3/0442; G06N 3/084

(52) U.S. Cl. CPC G16B 40/00; G06N 3/0442; G06N 3/084

(57) ABSTRACT

A method for RNA-protein interaction prediction, including: obtaining an RNA sequence to be predicted and a protein sequence to be predicted; obtaining a first RNA vector sequence by encoding the RNA sequence to be predicted; obtaining a first protein vector sequence by encoding the protein sequence to be predicted; obtaining a relevance vector sequence of the first RNA vector sequence and a relevance vector sequence of the first protein vector sequence through a selective attention mechanism model; and determining a probability value of interaction between the RNA sequence to be predicted and the protein sequence to be predicted according to the relevance vector sequence of the first RNA vector sequence and the relevance vector sequence of the first protein vector sequence.

### CROSS REFERENCE

The present disclosure is based upon International Application No. PCT/CN2021/134640, filed on Nov. 30, 2021, the entire contents of which are incorporated herein by reference.

### TECHNICAL FIELD

The present disclosure relates to the technical field of artificial intelligence, and in particular, to a method for RNA-protein interaction prediction, an apparatus for RNA-protein interaction prediction, a computer-readable storage medium, and an electronic device.

### BACKGROUND

Non-coding RNA (ncRNA) participates in many complex cell activity processes, plays an important role in the life processes such as selective shearing, chromatin modification, epigenetic inheritance and the like, and has a close relationship with many diseases. Researches show that most non-coding RNA realizes its regulation and control function by interacting with protein. Therefore, studying an interaction between the non-coding RNA and the protein has important significance for revealing the molecular action mechanism of the non-coding RNA in human diseases and life activities, and has become one of the important approaches for analyzing functions of the non-coding RNA and the protein at present.

It should be noted that the information disclosed in the above background part is only used to enhance the understanding of the background of the present disclosure, and therefore may include information that does not constitute the related art known to those of ordinary skill in the art.

### SUMMARY

The present disclosure provides a method for RNA-protein interaction prediction, an apparatus for RNA-protein interaction prediction, a computer-readable storage medium, and an electronic device.

The present disclosure provides a method for RNA-protein interaction prediction, including:
obtaining an RNA sequence to be predicted and a protein sequence to be predicted;
obtaining a first RNA vector sequence by encoding the RNA sequence to be predicted;
obtaining a first protein vector sequence by encoding the protein sequence to be predicted;
obtaining a relevance vector sequence of the first RNA vector sequence and a relevance vector sequence of the first protein vector sequence through a selective attention mechanism model; and
determining a probability value of interaction between the RNA sequence to be predicted and the protein sequence to be predicted according to the relevance vector sequence of the first RNA vector sequence and the relevance vector sequence of the first protein vector sequence.

In some embodiments of the present disclosure, obtaining a first RNA vector sequence by encoding the RNA sequence to be predicted includes:
converting the RNA sequence to be predicted into N base k-mer subsequences; and
obtaining the first RNA vector sequence by vectorizing each base k-mer subsequence.

In some embodiments of the present disclosure, the obtaining the first RNA vector sequence by vectorizing each base k-mer subsequence includes:
obtaining first vectors of N base k-mer subsequences by encoding each base k-mer subsequence;
obtaining second vectors of N base k-mer subsequences by performing an operation on the first vectors of the N base k-mer subsequences using a first mapping matrix;
composing the first RNA vector sequence with N base k-mer vectors by inputting the second vectors of the N base k-mer subsequences into a pre-trained recurrent neural network and outputting the N base k-mer vectors.

In some embodiments of the present disclosure, obtaining a first protein vector sequence by encoding the protein sequence to be predicted includes:
converting the protein sequence to be predicted into M amino acid k-mer subsequences; and
obtaining the first protein vector sequence by vectorizing each amino acid k-mer subsequence.

In some embodiments of the present disclosure, obtaining the first protein vector sequence by vectorizing each amino acid k-mer subsequence includes:
obtaining first vectors of M amino acid k-mer subsequences by encoding each amino acid k-mer subsequence;
obtaining second vectors of M amino acid k-mer subsequences by performing an operation on the first vectors of the M amino acid k-mer subsequences using a second mapping matrix; and
composing the first protein vector sequence with M amino acid k-mer vectors by inputting the second vectors of the M amino acid k-mer subsequences sequentially into a pre-trained recurrent neural network and outputting the M amino acid k-mer vectors.

In some embodiments of the present disclosure, obtaining a relevance vector sequence of the first RNA vector sequence and a relevance vector sequence of the first protein vector sequence through a selective attention mechanism model includes:
obtaining a first RNA hidden vector by performing feature extraction on the first RNA vector sequence;
obtaining a first protein hidden vector by performing feature extraction on the first protein vector sequence; and
obtaining the relevance vector sequence of the first RNA vector sequence and the relevance vector sequence of the first protein vector sequence by performing an operation on the first RNA hidden vector and the first protein hidden vector.

In some embodiments of the present disclosure, the first RNA hidden vector includes a first RNA vector, a second RNA vector, and a third RNA vector; and, obtaining a first RNA hidden vector by performing feature extraction on the first RNA vector sequence includes:
obtaining the first RNA vector by performing an operation on the first RNA vector sequence using a first query weight matrix;
obtaining the second RNA vector by performing an operation on the first RNA vector sequence using a first key weight matrix; and
obtaining the third RNA vector by performing an operation on the first RNA vector sequence using a first value weight matrix.

In some embodiments of the present disclosure, the first protein hidden vector includes a first protein vector, a second protein vector, and a third protein vector; and, obtaining a first protein hidden vector by performing feature extraction on the first protein vector sequence includes:
obtaining the first protein vector by performing an operation on the first protein vector sequence using a second query weight matrix;
obtaining the second protein vector by performing an operation on the first protein vector sequence using a second key weight matrix; and
obtaining the third protein vector by performing an operation on the first protein vector sequence using a second value weight matrix.

In some embodiments of the present disclosure, obtaining the relevance vector sequence of the first RNA vector sequence and the relevance vector sequence of the first protein vector sequence by performing an operation on the first RNA hidden vector and the first protein hidden vector includes:
obtaining a first RNA attention score by calculating a similarity between the first RNA vector and the second protein vector;
obtaining the relevance vector sequence of the first RNA vector sequence by summing the third protein vector according to the first RNA attention score;
obtaining a first protein attention score by calculating a similarity between the first protein vector and the second RNA vector; and
obtaining the relevance vector sequence of the first protein vector sequence by summing the third RNA vector according to the first protein attention score.

In some embodiments of the present disclosure, determining a probability value of interaction between the RNA sequence to be predicted and the protein sequence to be predicted according to the relevance vector sequence of the first RNA vector sequence and the relevance vector sequence of the first protein vector sequence includes:
obtaining a second RNA vector sequence and a second protein vector sequence according to the relevance vector sequence of the first RNA vector sequence and the relevance vector sequence of the first protein vector sequence; and
determining the probability value of interaction between the RNA sequence to be predicted and the protein sequence to be predicted according to the second RNA vector sequence and the second protein vector sequence.

In some embodiments of the present disclosure, obtaining a second RNA vector sequence and a second protein vector sequence according to the relevance vector sequence of the first RNA vector sequence and the relevance vector sequence of the first protein vector sequence includes:
obtaining an RNA fusion vector sequence by splicing the relevance vector sequence of the first RNA vector sequence and the first RNA vector sequence;
outputting the second RNA vector sequence by inputting the RNA fusion vector sequence into a pre-trained recurrent neural network;
obtaining a protein fusion vector sequence by splicing the relevance vector sequence of the first protein vector sequence and the first protein vector sequence; and
outputting the second protein vector sequence by inputting the protein fusion vector sequence into a pre-trained recurrent neural network.

In some embodiments of the present disclosure, obtaining a second RNA vector sequence and a second protein vector sequence according to the relevance vector sequence of the first RNA vector sequence and the relevance vector sequence of the first protein vector sequence includes:
obtaining an RNA fusion vector sequence by splicing the relevance vector sequence of the first RNA vector sequence and the first RNA vector sequence;
obtaining a protein fusion vector sequence by splicing the relevance vector sequence of the first protein vector sequence and the first protein vector sequence;
obtaining a self-relevance vector sequence of the RNA fusion vector sequence through a selective attention mechanism model and obtaining the second RNA vector sequence according to the self-relevance vector sequence of the RNA fusion vector sequence; and
obtaining a self-relevance vector sequence of the protein fusion vector sequence through a selective attention mechanism model and obtaining the second protein vector sequence according to the self-relevance vector sequence of the protein fusion vector sequence.

In some embodiments of the present disclosure, obtaining a self-relevance vector sequence of the RNA fusion vector sequence through a selective attention mechanism model and obtaining the second RNA vector sequence according to the self-relevance vector sequence of the RNA fusion vector sequence includes:
obtaining a second RNA hidden vector by performing feature extraction on the RNA fusion vector sequence;
obtaining the self-relevance vector sequence of the RNA fusion vector sequence by performing an operation on the second RNA hidden vector; and
obtaining the second RNA vector sequence by performing an operation on the self-relevance vector sequence of the RNA fusion vector sequence.

In some embodiments of the present disclosure, the second RNA hidden vector includes a fourth RNA vector, a fifth RNA vector, and a sixth RNA vector; and obtaining a second RNA hidden vector by performing feature extraction on the RNA fusion vector sequence includes:
obtaining the fourth RNA vector by performing an operation on the RNA fusion vector sequence using a third query weight matrix;
obtaining the fifth RNA vector by performing an operation on the RNA fusion vector sequence using a third key weight matrix; and
obtaining the sixth RNA vector by performing an operation on the RNA fusion vector sequence using a third value weight matrix.

In some embodiments of the present disclosure, obtaining the self-relevance vector sequence of the RNA fusion vector sequence by performing an operation on the second RNA hidden vector includes:
obtaining a second RNA attention score by calculating a similarity between the fourth RNA vector and the fifth RNA vector; and
obtaining the self-relevance vector sequence of the RNA fusion vector sequence by summing the sixth RNA vector according to the second RNA attention score.

In some embodiments of the present disclosure, obtaining a self-relevance vector sequence of the protein fusion vector sequence through a selective attention mechanism model and obtaining the second protein vector sequence according to the self-relevance vector sequence of the protein fusion vector sequence includes:
obtaining a second protein hidden vector by performing feature extraction on the protein fusion vector sequence;
obtaining the self-relevance vector sequence of the protein fusion vector sequence by performing an operation on the second protein hidden vector; and
obtaining the second protein vector sequence by performing an operation on the self-relevance vector sequence of the protein fusion vector sequence.

In some embodiments of the present disclosure, the second protein hidden vector includes a fourth protein vector, a fifth protein vector, and a sixth protein vector; and obtaining a second protein hidden vector by performing feature extraction on the protein fusion vector sequence includes:
obtaining the fourth protein vector by performing an operation on the protein fusion vector sequence using a fourth query weight matrix;
obtaining the fifth protein vector by performing an operation on the protein fusion vector sequence using a fourth key weight matrix; and
obtaining the sixth protein vector by performing an operation on the protein fusion vector sequence using a fourth value weight matrix.

In some embodiments of the present disclosure, obtaining the self-relevance vector sequence of the protein fusion vector sequence by performing an operation on the second protein hidden vector includes:
obtaining a second protein attention score by calculating a similarity between the fourth protein vector and the fifth protein vector; and
obtaining the self-relevance vector sequence of the protein fusion vector sequence by summing the sixth protein vector according to the second protein attention score.

In some embodiments of the present disclosure, determining the probability value of interaction between the RNA sequence to be predicted and the protein sequence to be predicted according to the second RNA vector sequence and the second protein vector sequence includes:
obtaining a feature vector to be predicted by splicing the second RNA vector sequence and the second protein vector sequence;
obtaining an interaction prediction value between the RNA sequence to be predicted and the protein sequence to be predicted according to the feature vector to be predicted; and
determining the probability value of interaction between the RNA sequence to be predicted and the protein sequence to be predicted according to the interaction prediction value.

In some embodiments of the present disclosure, obtaining an interaction prediction value between the RNA sequence to be predicted and the protein sequence to be predicted according to the feature vector to be predicted includes:
outputting a probability value of presence of interaction between the RNA sequence to be predicted and the protein sequence to be predicted by inputting the feature vector to be predicted into a classifier.

In some embodiments of the present disclosure, the method further includes:
obtaining a training data set, the training data set including a positive-example RNA-protein pair and a negative-example RNA-protein pair;
determining an interaction prediction value of each RNA-protein pair in the training data set by using a recurrent neural network and the selective attention mechanism model;
obtaining a corresponding loss value by calculating the interaction prediction value and a label value of each RNA-protein pair in the training data set using a loss function; and
adjusting model parameters of the recurrent neural network and the selective attention mechanism model according to the loss value.

In some embodiments of the present disclosure, the method further includes:
outputting an interaction prediction result between the RNA sequence to be predicted and the protein sequence to be predicted.

The present disclosure provides an apparatus for RNA-protein interaction prediction, including:
a data obtaining module, configured to obtain an RNA sequence to be predicted and a protein sequence to be predicted;
a first data encoding module, configured to obtain a first RNA vector sequence by encoding the RNA sequence to be predicted;
a second data encoding module, configured to obtain a first protein vector sequence by encoding the protein sequence to be predicted;
a relevance information obtaining module, configured to obtain a relevance vector sequence of the first RNA vector sequence and a relevance vector sequence of the first protein vector sequence through a selective attention mechanism model; and
an interaction determination module, configured to determine a probability value of interaction between the RNA sequence to be predicted and the protein sequence to be predicted according to the relevance vector sequence of the first RNA vector sequence and the relevance vector sequence of the first protein vector sequence.

The present disclosure provides a computer-readable storage medium, storing with a computer program thereon, where, when the computer program is executed by a processor, the method according to any one of the above is implemented.

The present disclosure provides an electronic device, including: a processor; and a memory, configured to store an executable instruction of the processor, where the processor is configured to execute the method according to any one of the above by executing the executable instruction.

It should be understood that the above general description and the following detailed description are merely exemplary and explanatory, and cannot limit the present disclosure.

### BRIEF DESCRIPTION OF THE DRAWINGS

The accompanying drawings, which are incorporated in and constitute a part of the description, illustrate embodiments consistent with the disclosure and together with the description serve to explain the principles of the disclosure. Obviously, the drawings in the following description are some embodiments of the present disclosure, and for those of ordinary skill in the art, other drawings can also be obtained from these drawings without creative efforts.

FIG. 1 shows a schematic diagram of an exemplary system architecture of a method and apparatus for RNA-protein interaction prediction according to some embodiments of the present disclosure;

FIG. 2 schematically shows a flowchart of a method for RNA-protein interaction prediction according to some embodiments of the present disclosure;

FIG. 3 schematically illustrates a flowchart of obtaining a first RNA vector sequence according to some embodiments of the present disclosure;

FIG. 4 schematically shows a flowchart of obtaining a first protein vector sequence according to some embodiments of the present disclosure;

FIG. 5 schematically shows a flowchart of obtaining relevance information between an RNA sequence and a protein sequence according to some embodiments of the present disclosure;

FIG. 6 schematically shows a flowchart of obtaining a second RNA vector sequence and a second protein vector sequence according to some embodiments of the present disclosure;

FIG. 7 schematically shows a flowchart of obtaining a second RNA vector sequence according to some embodiments of the present disclosure;

FIG. 8 schematically shows a flowchart of obtaining a second protein vector sequence according to some embodiments of the present disclosure;

FIG. 9 schematically shows a flowchart of determining a probability value of interaction between an RNA sequence and a protein sequence according to some embodiments of the present disclosure;

FIG. 10 schematically shows a flowchart of model training according to some embodiments of the present disclosure;

FIG. 11 schematically shows a flowchart of a method for RNA-protein interaction prediction according to some embodiments of the present disclosure;

FIG. 12 schematically shows a block diagram of an apparatus for RNA-protein interaction prediction according to some embodiments of the present disclosure;

FIG. 13 is a schematic structural diagram of a computer system adaptable to implement an electronic device according to some embodiments of the present disclosure.

### DETAILED DESCRIPTION

Example embodiments will now be described more fully with reference to the accompanying drawings. Example embodiments, however, can be implemented in various forms and should not be construed as limited to the embodiments set forth here; by contrast, these embodiments are provided so that the present disclosure will be thorough and complete, and will fully convey the concept of example embodiments to those skilled in the art. The described features, structures, or characteristics may be combined in any suitable manner in one or more embodiments. In the following description, numerous specific details are provided to give a thorough understanding of embodiments of the present disclosure. However, those skilled in the art will appreciate that the technical solutions of the present disclosure may be practiced, while omitting one or more of the specific details, or employing other methods, components, apparatuses, steps, etc. In other instances, commonly known technical solutions are not shown or described in detail to avoid obscuring aspects of the present disclosure.

In addition, the drawings are merely schematic illustrations of the present disclosure, and are not necessarily drawn to scale. Same reference numerals in the drawings denote the same or similar parts, and thus repeated descriptions of them will be omitted. Some block diagrams shown in the drawings are functional entities, and do not necessarily correspond to physical or logically independent entities. These functional entities may be implemented in the form of software, or in one or more hardware modules or integrated circuits, or in different networks and/or processor devices and/or microcontroller devices.

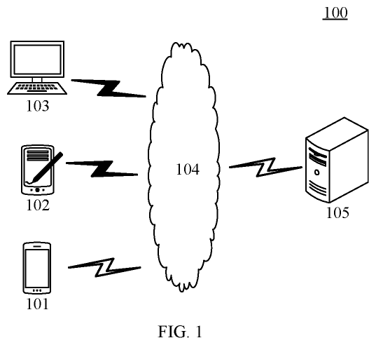

FIG. 1 shows a schematic diagram of a system architecture of an exemplary application environment of a method and apparatus for RNA-protein interaction prediction according to some embodiments of the present disclosure.

As shown in FIG. 1, the system architecture 100 of the interaction prediction system may include one or more of terminal devices 101, 102, 103, and network 104 and server 105. Network 104 is configured to provide a medium for a communication link between terminal devices 101, 102, 103 and server 105. Network 104 may include various connection types, such as wired or wireless communication links, fiber optic cables, and the like. Terminal devices 101, 102, 103 may be various electronic devices, including but not limited to desktop computers, portable computers, smartphones, tablet computers, and the like. It should be understood that the number of terminal devices, networks, and servers in FIG. 1 is merely illustrative. According to implementation requirements, there may be any number of terminal devices, networks, and servers. For example, server 105 may be one server, or may be a server cluster composed of a plurality of servers, or may be a cloud computing platform or a virtualization center. Specifically, server 105 may be configured to: obtain an RNA sequence to be predicted and a protein sequence to be predicted; obtain a first RNA vector sequence by encoding the RNA sequence to be predicted; obtain a first protein vector sequence by encoding the protein sequence to be predicted; and obtain a relevance vector sequence of the first RNA vector sequence and a relevance vector sequence of the first protein vector sequence through a selective attention mechanism model; and determine a probability value of interaction between the RNA sequence to be predicted and the protein sequence to be predicted according to the relevance vector sequence of the first RNA vector sequence and the relevance vector sequence of the first protein vector sequence.

The method for RNA-protein interaction prediction provided in the embodiments of the present disclosure is generally executed by server 105, and correspondingly, the apparatus for RNA-protein interaction prediction is generally disposed in server 105. The server may send an interaction prediction result between the RNA sequence to be predicted and the protein sequence to be predicted to the terminal device, which is displayed to the user by the terminal device. It would be easy for those skilled in the art to understand that the method for RNA-protein interaction prediction provided in the embodiments of the present disclosure may also be executed by one or more of terminal devices 101, 102, 103, and correspondingly, the apparatus for RNA-protein interaction prediction may also be disposed in terminal devices 101, 102, 103. For example. After being executed by the terminal device, the prediction result may be directly displayed on the display screen of the terminal device, or may be provided to the user in a voice broadcast manner, which is not specifically limited in the exemplary embodiments.

The technical solutions of the embodiments of the present disclosure are described in detail below.

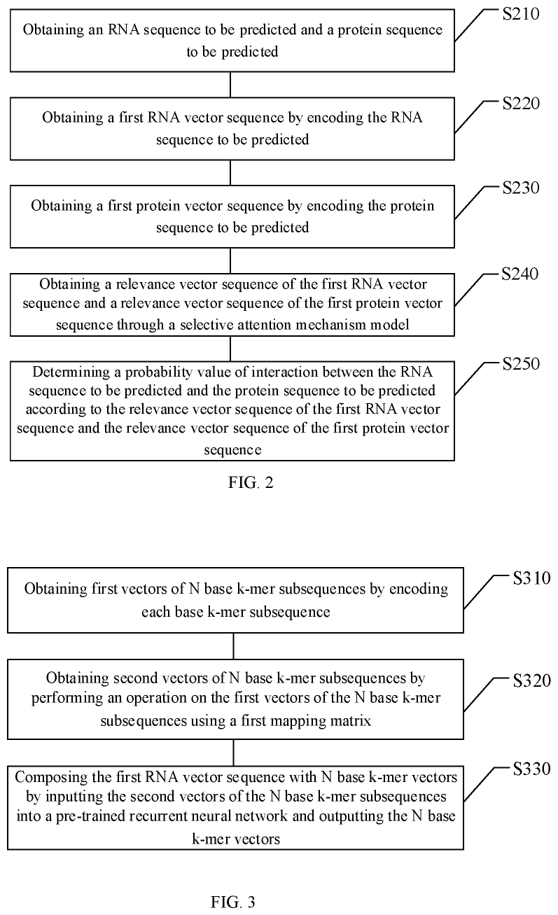

This example embodiment provides a method for RNA-protein interaction prediction, which may be applied to the above-mentioned server 105, or may be applied to one or more of the above-mentioned terminal devices 101, 102, and 103, which is not specifically limited in this example embodiment. Referring to FIG. 2, the method for RNA-protein interaction prediction may include the following steps S210 to S250.

In step S210, an RNA sequence to be predicted and a protein sequence to be predicted are obtained.

In step S220, a first RNA vector sequence is to obtained by encoding the RNA sequence to be predicted;

In step S230, a first protein vector sequence is to obtained by encoding the protein sequence to be predicted;

In step S240, a relevance vector sequence of the first RNA vector sequence and a relevance vector sequence of the first protein vector sequence are obtained through a selective attention mechanism model;

In step S250, a probability value of interaction between the RNA sequence to be predicted and the protein sequence to be predicted is determined according to the relevance vector sequence of the first RNA vector sequence and the relevance vector sequence of the first protein vector sequence.

In the method for RNA-protein interaction prediction provided by an example embodiment of the present disclosure, an RNA sequence to be predicted and a protein sequence to be predicted are obtained; a first RNA vector sequence is to obtained by encoding the RNA sequence to be predicted; a first protein vector sequence is to obtained by encoding the protein sequence to be predicted; a relevance vector sequence of the first RNA vector sequence and a relevance vector sequence of the first protein vector sequence are obtained through a selective attention mechanism model; and a probability value of interaction between the RNA sequence to be predicted and the protein sequence to be predicted is determined according to the relevance vector sequence of the first RNA vector sequence and the relevance vector sequence of the first protein vector sequence. According to the present disclosure, the relevance information between the RNA sequence and the protein sequence is determined by using the selective attention mechanism model; the RNA sequence, the protein sequence and the relevance information can be fused; and when predicting the probability value of interaction between the RNA sequence and the protein sequence by introducing the sequence information obtained by fusion, the accuracy of interaction prediction between the RNA and the protein can be improved.

The above steps in this embodiment of the present example are described in more detail below.

In step S210, an RNA sequence to be predicted and a protein sequence to be predicted are acquired.

In this example embodiment, at least one RNA-protein pair to be predicted consisting of an RNA sequence and a protein sequence can be obtained, and the probability value of interaction between the RNA sequence and the protein sequence in each RNA-protein pair to be predicted is unknown. For example, the user may input the RNA-protein pair to be predicted through the terminal device. For example, the user may manually input the RNA-protein pair to be predicted, or may input the RNA-protein pair to be predicted through voice, which is not specifically limited in this example. For example, it may be that an RNA sequence is inputted, and then a protein sequence is inputted, where the input order of the two is not limited. For example, the RNA sequence and the protein sequence may be input into different text boxes, or may be input into the same text box. For example, after the input is completed, the “start prediction” button is clicked, and then the prediction steps provided in some embodiments of the present application start to be performed.

Among them, the interaction between the RNA and the protein means that functions of the protein are embodied in the interaction with other proteins and the RNA. For example, the interaction between the protein and the RNA plays an important role in synthesis of the protein. At the same time, the realization of many functions of RNA also relies on the interaction with the protein. The interaction may be a regulation and control function, a guidance function, and the like, which is not limited here. For example, in the presence of interaction, the RNA can guide synthesis of the protein, or the RNA can regulate and control the realization of functions of the protein. The interaction between the RNA and the protein may also mean that the RNA and the protein may adjust the life cycle and functions of each other through physical interaction. For example, the RNA coding sequence can guide synthesis of the protein, and correspondingly, the protein can also regulate expression and functions of the RNA.

After obtaining the RNA-protein pair to be predicted, the interaction prediction system may be used to predict the probability value of interaction in each input RNA-protein pair to be predicted, and determine whether an interaction is present in each RNA-protein pair to be predicted according to the prediction result. At the same time, the interaction prediction result in the RNA-protein pair to be predicted may also be output to the terminal device for the user to view. For example, the prediction result may be directly displayed on the display screen of the terminal device, or may be provided to the user in a voice broadcast manner, which is not specifically limited in this example.

In other examples, at least one RNA sequence to be predicted may also be obtained, and a protein sequence interacting with each input RNA sequence to be predicted is searched in a database. For example, after the user inputs the RNA sequence to be predicted through the terminal device, at least one protein sequence in the database can be selected, and a plurality of RNA-protein pairs are composed by the RNA sequence to be predicted and each protein sequence, so that the a probability value of interaction of each RNA-protein pair can be predicted through the interaction prediction system, and a protein sequence that can interact with the RNA sequence to be predicted is output according to the prediction result. In some embodiments, several types of protein sequences may be pre-stored in a database so as to be invoked when predicting the probability value of interaction of the RNA-protein pairs. For example, the protein sequence may be stored in a Redis database, or may be stored in a MySQL database, so that a protein sequence to be predicted may be queried and selected in real time. Among them, Redis is a key-value storage system. When stored in a Redis database, the protein sequence may include a sequence identification (such as a sequence number) and a key-value pair formed by a corresponding protein sequence, where the key is a sequence identification, and the value is a corresponding protein sequence. Redis is used as an efficient caching technology. Redis can support a read-write frequency exceeding 100 K+ per second, and has a certain advantage in data reading and storage speed. MySQL is a relevance database management system, the association database stores data in different tables instead of uniformly storing all data, increasing storage speed and improving flexibility, having a stable advantage in data storage, and avoiding data loss.

It can be understood that several types of RNA sequences may also be pre-stored in a database so as to be invoked when predicting the probability value of interaction of the RNA-protein pair. Therefore, at least one protein sequence to be predicted may also be obtained, and an RNA sequence interacting with each input protein sequence to be predicted is searched in the database. Similarly, after the user inputs the protein sequence through the terminal device, at least one RNA sequence in the database can be selected, and a plurality of RNA-protein pairs are composed by the protein sequence to be predicted and each RNA sequence, so that the probability value of interaction of each RNA-protein pair can be predicted through the interaction prediction system, and an RNA sequence that can interact with the protein sequence to be predicted is output according to the prediction result, which is not specifically limited in the present disclosure.

In step S220, a first RNA vector sequence is obtained by encoding the RNA sequence to be predicted.

After obtaining the RNA sequence to be predicted, the RNA sequence to be predicted may be encoded to obtain a first RNA vector sequence, so as to obtain relevance information between the first RNA vector sequence and the first protein vector sequence, thus predicting the probability value of interaction between the RNA sequence and the protein sequence based on the relevance information.

In an example embodiment of the present disclosure, an RNA sequence may be represented by a base sequence, for example, one RNA sequence may be represented as AGCAUCAGCCU . . . For an RNA sequence, four bases may be included, which are adenine (A), uracil (U), guanine (G), and cytosine (C), respectively. Correspondingly, a base k-mer subsequence may also be used to represent an RNA sequence. Among them, the k-mer subsequence refers to a k-linked body composed of k bases or k-type of amino acids as a group. Specifically, four bases may be arranged and combined to obtain all base k-mer subsequences, and 4k base k-mer subsequences may be obtained for a certain k value. For example, when k is 3, there is a total of 4 3=64 base 3-mer subsequences, and when k is 4, there is a total of 4 4=256 base 4-mer subsequences. For example, AGC, AUA, GCA, and CCU are four different base 3-mer subsequences, and AGCA, UAGC, and ACCU are three different base 4-mer subsequences. Therefore, the RNA sequence AGCAUCAGCCU... may also be represented as {AGC, AUA, GCA, CCU, . . . }, and may also be represented as {AGCA, UAGC, ACCU, ...}. In other examples, the RNA sequence may also be read in an overlapping manner to obtain a corresponding base 3-mer subsequence or a base 4-mer subsequence. Correspondingly, the base 3-mer subsequence of the RNA sequence may also include AGC, GCA, CAU, AUA, etc. The base 4-mer subsequence of the RNA sequence may also include AGCA, GCAU, CAUA, etc., which is not specifically limited in the present disclosure. In an example embodiment of the present disclosure, k is a positive integer, for example, 1, 2, 3 . . . . The k value can take one or more values, and the specific value of k may be adjusted according to actual situations, which is not limited here.

When the RNA sequence to be predicted is encoded, a part of the bases of the RNA sequence to be predicted can be encoded, and the encoding result is used as a first RNA vector sequence. All bases of the RNA sequence to be predicted can also be encoded, and all encoded bases compose the first RNA vector sequence. All bases of the RNA sequence to be predicted can also be encoded, and a part of encoded bases are selected to compose the first RNA vector sequence, which is not specifically limited in the present disclosure.

In an example embodiment of the present disclosure, it is illustrated by taking that all bases of an RNA sequence to be predicted are encoded and all encoded bases compose a first RNA vector sequence as an example. The RNA sequence to be predicted may be converted into N base k-mer subsequences. For example, according to the value of k, continuous k bases can be sequentially taken from the first base of the RNA sequence to be predicted to compose a base k-mer subsequence of the RNA sequence to be predicted, until the last k bases in the RNA sequence to be predicted are taken up, and all base k-mer subsequences of the RNA sequence to be predicted are obtained. Then, each base k-mer subsequence may be vectorized to obtain N base k-mer vectors, and the N base k-mer vectors compose a first RNA vector sequence. For example, the RNA sequence to be predicted may be divided into N base k-mer subsequences without overlapping. For example, if the RNA sequence to be predicted is AUCUGAAU, the RNA sequence to be predicted may be divided into three base k-mer subsequences, which are AUC, UGA, and AAU, respectively. It can be understood that an RNA sequence is divided into a plurality of base k-mer subsequences without overlapping, so that the bases in an RNA sequence is vectorized and represented in the form of k-linked body. Similarly, in other examples, each base included in the RNA sequence to be predicted may also be vectorized to obtain a plurality of base vectors, and the plurality of base vectors compose a first RNA vector sequence. The RNA sequence to be predicted may also be divided into P base k-mer subsequences with overlapping, each base k-mer sequence is vectorized to obtain P base k-mer vectors, and the P base k-mer vectors compose a first RNA vector sequence, which is not specifically limited in the present disclosure.

In an example embodiment, after the RNA sequence to be predicted is converted into N base k-mer subsequences, each base k-mer subsequence in the RNA sequence to be predicted can be encoded to obtain a first vector of the N base k-mer subsequences, and the first vector of the N base k-mer subsequences compose a first RNA vector sequence.

In some embodiments of the present disclosure, for a certain k value, there may be 4k base k-mer subsequences, and One-Hot encoding may be performed on each base k-mer subsequence. Among them, One-Hot encoding is also referred to as one-bit-effective encoding, and the method is to use an N-bit state register to encode N states, each state having an independent register bit, and at any time, only one bit is effective in the register. For example, when k=3, there may be 64 base 3-mer subsequences, and One-Hot encoding may be performed on each base 3-mer subsequence to obtain a first vector of the base k-mer subsequence.

For example, for the i-th base 3-mer subsequence, that is, the base 3-mer subsequence with an index of an integer i, a 64-dimensional One-Hot vector may be obtained through encoding. The i-th element in the vector is set to 1, and the other elements are all set to 0, such as [0, 1, 0, 0. 0]. Similarly, each base 3-mer subsequence may correspond to a base 3-mer One-Hot vector. For another example, when k=1, each base is a base 1-mer subsequence, that is, each base in the RNA sequence to be predicted may be encoded to obtain a representation vector corresponding to each base. For example, if the RNA sequence to be predicted includes L bases, for the j-th base, that is, a base with an index of an integer j, an L-dimensional One-Hot vector may be obtained through encoding. The j-th element in the vector is set to 1, and the other elements are all set to 0, to obtain the One-Hot vector of the j-th base. In other examples, each base in the RNA sequence to be predicted may also be encoded into a 4-dimensional One-Hot vector according to the base type. For example, base A may be represented by One-Hot vector [1, 0, 0, 0], U is represented as [0, 0, 0, 1], G is represented as [0, 1, 0, 0], and C is represented as [0, 0, 1, 0]. Correspondingly, the One-Hot vector of each base in the RNA sequence to be predicted may be obtained.

For example, for an RNA sequence AUCUGAAAU to be predicted, three base 3-MER subsequences of AUC, UGA and AAU may be included, and the corresponding three base 3-mer One-Hot vectors are V₁^{R}, V₂^{R} and V₃^{R} respectively. Three base 3-mer One-Hot vectors may compose a first RNA vector sequence {V₁^{R}V₂^{R}, V₃^{R}}. In an example embodiment of the present disclosure, by performing One-Hot encoding on the base k-mer subsequence, each base k-mer subsequence may be changed into a binary feature, thus making up for the defects when the classifier processes attribute data, so as to more accurately predict the probability value of interaction between the RNA sequence and the protein sequence using the classifier.

In some embodiments of the present disclosure, each base k-mer subsequence may be represented by using a dense vector. That is, Embedding (vector mapping) encoding is performed on each base k-mer subsequence sequentially, each base k-mer subsequence is respectively represented by a low-dimensional vector to obtain a plurality of corresponding base k-mer Embedding vectors, and the plurality of base k-mer Embedding vectors compose a first RNA vector sequence. For example, each base k-mer subsequence in an RNA sequence may be mapped into a vector space by using a Word2vec algorithm, and each base k-mer subsequence may be represented by a vector in the vector space. The base k-mer subsequence may also be converted into an Embedding vector by using a Doc2vec algorithm, a Glove algorithm, etc. Each base k-mer subsequence may also be encoded by using a BERT (bidirectional encoder representations from transformer) pre-training model to obtain a plurality of corresponding base k-mer embedding vectors, which is not specifically limited in the present disclosure. In an example embodiment of the present disclosure, by performing Embedding encoding on the base k-mer subsequence, the discrete base k-mer subsequences may be converted into a low-dimensional continuous vector, and each base k-mer subsequence may be better represented by using the continuous vector. Moreover, the Embedding encoding process is learnable, and in the continuous training process, similar base k-mer subsequences can be closer in the vector space, and category differentiation is performed while encoding the base k-mer subsequence, so that the probability value of interaction between the RNA sequence and the protein sequence can be more accurately predicted subsequently. In addition, the prediction efficiency of the probability value of interaction is also improved to a certain extent.

In an example embodiment, after the RNA sequence to be predicted is converted into N base k-mer subsequences, each base k-mer subsequence may be encoded to obtain first vectors of the N base k-mer subsequences. The first vectors of the N base k-mer subsequences are input into a pre-trained recurrent neural network sequentially, N base k-mer vectors are output, and the N base k-mer vectors compose a first RNA vector sequence.

For example, the first vector may be a One-Hot vector. It can be understood that there is a connection between the various bases in the RNA sequence. In this example, all base k-mer One-Hot vectors in the RNA sequence to be predicted may be regarded as a time sequence sequence, and then an operation may be performed on each base k-mer One-Hot vector by using a recurrent neural network. For example, after all base 3-mer One-Hot vectors (V₁^{R}, V₂^{R} and V₃^{R}) in the RNA sequence AUCUGAAAU to be predicted are obtained, the three base 3-mer One-Hot vectors can be input into a trained LSTM network, and each corresponding base 3-mer vector is output, which is h₁^{R}, h₂^{R} and h₃^{R}, respectively. The three base 3-mer vectors compose a first RNA vector sequence {h₁^{R}, h₂^{R}, h₃^{R}}, where the LSTM network is a time recurrent neural network, which is suitable for processing and predicting an important event with a relatively long interval and delay in a time sequence.

In an example embodiment, after the RNA sequence to be predicted is converted into N base k-mer subsequences, each base k-mer subsequence may be encoded to obtain first vectors of the N base k-mer subsequences. An operation (for example, a product operation) is performed on the first vectors of the N base k-mer subsequences by using a first mapping matrix to obtain second vectors of the N base k-mer subsequences, and the second vectors of the N base k-mer subsequences composes a first RNA vector sequence.

For example, the first vector may be a One-Hot vector, and the second vector may be an Embedding vector. For the RNA sequence AUCUGAAAU to be predicted, it may include three base 3-mer subsequences of AUC, UGA and AAU. One-Hot encoding may be performed on each base 3-mer subsequence to obtain the base 3-mer One-Hot vectors, which are V₁^{R}, V₂^{R} and V₃^{R}, respectively. Since the base 3-mer One-Hot vector is a 64-dimensional sparse vector, the base 3-mer One-Hot vector may be mapped to a dense Embedding vector through a first mapping matrix Wi. That is, according to the following:

Eᵢ^{R}=W₁×Vᵢ^{R} (1),

the i-th base 3-mer Embedding vector Eₑ^{R} in the RNA sequence to be predicted is obtained, where Vᵢ^{R} represents the i-th base 3-mer One-Hot vector in the RNA sequence to be predicted, and the first mapping matrix W₁ is a A*64 parameter matrix. For example, A may be 128 or 256, and the value of A is not specifically limited in the present disclosure. Based on this, the 3-mer embedding vectors corresponding to the three base 3-mer subsequences can be obtained in sequence, which are E₁^{R}, E₂^{R} and Eₑ^{R}, respectively, and then the three base 3-mer Embedding vectors compose a first RNA vector sequence.

In an example embodiment, after the RNA sequence to be predicted is converted into N base k-mer subsequences, referring to FIG. 3, each base k-mer subsequence may be encoded according to steps S310 to S330 to obtain a first RNA vector sequence.

In step S310, first vectors of N base k-mer subsequences are obtained by encoding each base k-mer subsequence.

For example, the first vector may be a One-Hot vector. For the RNA sequence AUCUGAAAU to be predicted, it may include three base 3-mer subsequences of AUC, UGA and AAU. One-Hot encoding may be performed on each base 3-mer subsequence to obtain the base 3-mer One-Hot vectors, which are V₁^{R}, V₂^{R} and V₃^{R} respectively.

In step S320, second vectors of the N base k-mer subsequences are obtained by performing an operation on the first vectors of the N base k-mer subsequences using a first mapping matrix.

The second vector may be an Embedding vector. Since the base 3-mer One-Hot vector is a 64-dimensional sparse vector, the base 3-mer One-Hot vector may be mapped to a dense Embedding vector through the first mapping matrix W₁ to obtain three base 3-mer Embedding vectors, which are E₁^{R}, E₂^{R} and E₃^{R}, respectively.

In step S330, the second vectors of the N base k-mer subsequences are sequentially input into a pre-trained recurrent neural network, N base k-mer vectors are output, and the N base k-mer vectors compose the first RNA vector sequence.

It can be understood that there is a connection between the various bases in the RNA sequence. In this example, all base 3-mer Embedding vectors in the RNA sequence to be predicted may be regarded as a time sequence sequence, so that an operation may be performed on each base 3-mer Embedding vector by using a recurrent neural network. For example, after all base 3-mer Embedding vectors (E₁^{R}, E₂^{R} and E₃^{R}) in the RNA sequence AUCUGAAAU to be predicted are obtained, the three base 3-mer Embedding vectors can be sequentially input into the trained LSTM network, and each corresponding base 3-mer vector is output, which is V₁^{R}, V₂^{R} and V₃^{R}, respectively. The three base 3-mer vectors compose a first RNA vector sequence.

Specifically, the Embedding vector E₁^{R} corresponding to “AUC” may be firstly input into the LSTM network. Hidden feature extraction may be performed on E₁^{R} through the LSTM network, and a hidden vector h₁^{R} at that moment, such as moment t, may be output. Then, the hidden vector h₁^{R} at moment t may be spliced with the Embedding vector E₂^{R} a corresponding to “UGA” at moment t+1, and the spliced vector is input into the LSTM network. Hidden feature extraction may be performed on the spliced vector to output the hidden vector h₂^{R} at moment t+1. Similarly, the Embedding vector at the current moment can be sequentially spliced with the hidden vector passed down from the previous moment, and feature extraction can be performed on the spliced vector through the LSTM network. Finally, the Embedding vector E₃^{R} corresponding to “AAU” can be input into the LSTM network, and the hidden vector at moment t+1 is spliced with the Embedding vector E₃^{R}. Hidden feature extraction is performed on the spliced vector through the LSTM network, and the hidden feature h₃^{R} at the last moment is output. In other examples, the operation can be performed on each base 3-mer Embedding vector by using a GRU network. The structure of the GRU network is relatively simple, and the implementation effect is the same as that of the LSTM network. Each base 3-mer One-Hot vector in the RNA sequence to be predicted may also be directly input into the GRU network to obtain a corresponding base 3-mer vector, which is not specifically limited in the present disclosure.

In this embodiment, when processing the plurality of base 3-mer Embedding vectors in the RNA sequence to be predicted using the LSTM network, the dependency relationship between various base 3-mer Embedding vectors can be learned and memorized. Based on this, the relevance information between the RNA sequence and the protein sequence can be obtained more accurately, and the probability value of interaction between the RNA sequence and the protein sequence is accurately predicted by fusing the relevance information.

In step S230, a first protein vector sequence is to obtained by encoding the protein sequence to be predicted.

After obtaining the protein sequence to be predicted, the protein sequence to be predicted can be encoded to obtain a first protein vector sequence, so as to obtain relevance information between the first RNA vector sequence and the first protein vector sequence, thus predicting the probability value of interaction between the RNA sequence and the protein sequence based on the relevance information.

In an example embodiment, a protein sequence may be represented by an amino acid sequence. It may include 20 amino acids, which are sequentially encoded as A, G, V, I, L, F, P, Y, M, T, S, H, N, Q, W, R, K, D, E, and C. For example, a protein sequence may be represented as MTAQDDSYS . . . . Correspondingly, an amino acid k-mer subsequence may also be used to represent a protein sequence. Specifically, 20 amino acids can be arranged and combined to obtain all amino acid k-mer subsequences. 20 k amino acid k-mer subsequences can be obtained for a certain k value. For example, when k is 3, there is a total of 20 3=8000 amino acid 3-mer subsequences. For example, MTA, QDD and SYS are three different amino acid 3-mer subsequences. Therefore, the protein sequence MICHQDDSYS . . . may also be represented as {MTA, QDD, SYS, . . . }. In other examples, the protein sequence may also be read in an overlapping manner to obtain corresponding amino acid 3-mer subsequences. Correspondingly, the amino acid 3-mer subsequences of the protein sequence may also include MTA, TAQ, AQD, etc. According to the physicochemical properties of amino acids, 20 amino acids may also be classified into seven types of {A, G, V}, {I, L, F, P}, {Y, M, T, S}, {H, N, Q, W}, {R, K}, {D, E}, and {C}, and each type of amino acid is encoded, for example, being encoded as 1, 2, 3, 4, 5, 6, and 7 in sequence. For example, the protein sequence MTAQDDSYS . . . may be converted into 331466333 . . . . Then, 7 types of amino acids can be arranged and combined to obtain all amino acid k-mer subsequences. 7 k amino acid k-mer subsequences can be obtained for a certain k value, which is not specifically limited in the present disclosure. It can be understood that it is merely indicative to classify 20 amino acids into 7 types, and 20 amino acids may also be classified according to the constituent components of the amino acids. Similarly, four bases of the RNA sequence may also be classified according to actual needs.

When encoding the protein sequence to be predicted, a part of amino acids of the protein sequence to be predicted can be encoded, and the encoding result is used as a first protein vector sequence. All amino acids of the protein sequence to be predicted can also be encoded, and all encoded amino acids compose the first protein vector sequence. All amino acids of the protein sequence to be predicted can also be encoded, and a part of encoded amino acids are selected to compose the first protein vector sequence, which is not specifically limited in the present disclosure.

In an example embodiment of the present disclosure, it is illustrated by taking that all amino acids of the protein sequence to be predicted are encoded and all encoded amino acids compose the first vector sequence as an example. The protein sequence to be predicted may be converted into M amino acid k-mer subsequences. For example, according to the value of k, continuous k amino acid can be sequentially taken from the first amino acid of the protein sequence to be predicted to compose an amino acid k-mer subsequence of the protein sequence to be predicted, until the last k amino acids in the protein sequence to be predicted are taken up, and all amino acid k-mer subsequences of the protein sequence to be predicted are obtained. Then, each amino acid k-mer subsequence may be vectorized to obtain M amino acid k-mer vectors, and the M amino acid k-mer vectors compose a first protein vector sequence. For example, a protein sequence to be predicted may be divided into M amino acid k-mer subsequences without overlapping. For example, if the protein sequence to be predicted is MTAQDDSYS, the protein sequence to be predicted may be divided into three amino acid k-mer subsequences, which are MTA, QDD, and SYS, respectively. Similarly, in other examples, each amino acid included in the protein sequence to be predicted may also be vectorized to obtain a plurality of amino acid vectors, and the plurality of amino acid vectors compose a first protein vector sequence. The protein sequence to be predicted may also be divided into Q amino acid k-mer subsequences with overlapping, each amino acid k-mer subsequence is vectorized to obtain Q amino acid k-mer vectors, and the Q amino acid k-mer vectors compose a first protein vector sequence, which is not specifically limited in the present disclosure.

In an example embodiment, after the protein sequence to be predicted is converted into M amino acid k-mer subsequences, each amino acid k-mer subsequence in the protein sequence to be predicted can be encoded to obtain first vectors of the M amino acid k-mer subsequences, and the first vectors of the M amino acid k-mer subsequences compose a first protein vector sequence. For example, when k=3, there may be 8000 amino acid 3-mer subsequences, and One-Hot encoding may be performed on each amino acid 3-mer subsequence to obtain a first vector of the amino acid k-mer subsequence.

For example, for the j-th amino acid 3-mer subsequence, that is, the amino acid 3-mer subsequence with an index of an integer j, an 8000-dimensional One-Hot vector may be obtained through encoding. The j-th element in the vector is set to 1, and the other elements are all set to 0, such as [1, 0, 0. 0]. Similarly, each amino acid 3-mer subsequence may correspond to an amino acid 3-mer One-Hot vector. For another example, when k=1, each amino acid is an amino acid 1-mer subsequence, that is, each amino acid in the protein sequence to be predicted may be encoded to obtain a representation vector corresponding to each amino acid. For example, if the protein sequence to be predicted includes S amino acids, for the j-th amino acid, that is, the amino acid with an index of an integer j, an S-dimensional One-Hot vector may be obtained through encoding. The j-th element in the vector is set to 1, and the other elements are all set to 0, to obtain the One-Hot vector of the j-th amino acid. In other examples, each amino acid in the protein sequence to be predicted may also be encoded into a 20-dimensional One-Hot vector according to the amino acid type, so as to obtain the One-Hot vector of each amino acid in the protein sequence to be predicted. 20 amino acids may also be classified, and each amino acid in the protein sequence to be predicted is encoded into a One-Hot vector having a vector dimension consistent with the number of classification categories. For example, when 20 amino acids are classified into seven categories, each amino acid in the protein sequence to be predicted may be encoded into a 7-dimensional One-Hot vector, which is not specifically limited in the present disclosure.

For example, for a protein sequence MTAQDDSYS to be predicted, it may include three amino acid 3-MER subsequences of MTA, QDD and SYS, and the corresponding three amino acid 3-mer One-Hot vectors are V₁^{P}, V₂^{P} and V₃^{P}, respectively. Three amino acid 3-mer One-Hot vectors may compose the first protein vector sequence {V₁^{P}, V₂^{P}, V₃^{P}}. In an example embodiment of the present disclosure, by performing One-Hot encoding on the amino acid k-mer subsequence, each amino acid k-mer subsequence may be changed into a binary feature, thus making up for the defects when the classifier processes the attribute data, so as to more accurately predict the probability value of interaction between the RNA sequence and the protein sequence using the classifier.

In some embodiments of the present disclosure, each amino acid k-mer subsequence may be represented by using a dense vector. That is, Embedding encoding is performed on each amino acid k-mer subsequence sequentially, each amino acid k-mer subsequence is respectively represented by a low-dimensional vector to obtain a plurality of corresponding amino acid k-mer Embedding vectors, and the plurality of amino acid k-mer Embedding vectors compose the first protein vector sequence. For example, each amino acid k-mer subsequence in a protein sequence may be mapped into a vector space by using a Word2vec algorithm, and each amino acid k-mer subsequence may be represented by a vector in the vector space. The amino acid k-mer subsequence may also be converted into an Embedding vector by using a Doc2vec algorithm, a Glove algorithm, etc. Each amino acid k-mer subsequence may also be encoded by using a BERT pre-training model to obtain a plurality of corresponding amino acid k-mer embedding vectors, which is not specifically limited in the present disclosure. In an example embodiment of the present disclosure, by performing Embedding encoding on the amino acid k-mer subsequence, the discrete amino acid k-mer subsequences may be converted into a low-dimensional continuous vector, and each amino acid k-mer subsequence may be better represented by using the continuous vector. Moreover, the Embedding encoding process is learnable, and in the continuous training process, similar amino acid k-mer subsequences can be closer in the vector space, and category differentiation is performed while encoding the amino acid k-mer subsequence, so that the probability value of interaction between the RNA sequence and the protein sequence can be more accurately predicted subsequently. In addition, the prediction efficiency of the probability value of interaction is also improved to a certain extent.

In an example embodiment, after the protein sequence to be predicted is converted into M amino acid k-mer subsequences, each amino acid k-mer subsequence can be encoded to obtain first vectors of the M amino acid k-mer subsequences. The first vectors of the M amino acid k-mer subsequences are input into a pre-trained recurrent neural network sequentially, M amino acid k-mer vectors are output, and the M amino acid k-mer vectors compose a first protein vector sequence.

For example, the first vector may be a One-Hot vector. It can be understood that there is a connection between the various amino acids in the protein sequence. In this example, all amino acid 3-mer One-Hot vectors in the protein sequence to be predicted can be regarded as a time sequence sequence, and then an operation may be performed on each amino acid 3-mer One-Hot vector by using a recurrent neural network. For example, after all amino acid 3-Mer One-Hot vectors (V₁^{P}, V₂^{P} and V₃^{P}) in the protein sequence MTAQDDSYS to be predicted are obtained, the three amino acid 3-Mer One-Hot vectors can be sequentially input into the trained LSTM network, and each corresponding amino acid 3-mer vector is output, which is h₁^{P}, h₂^{P} and h₃^{P}, respectively. The three amino acid 3-mer vectors compose a first protein vector sequence {h₁^{P}, h₂^{P}, h₃^{P}}.

In an example embodiment, after the protein sequence to be predicted is converted into M amino acid k-mer subsequences, each amino acid k-mer subsequence can be encoded to obtain first vectors of the M amino acid k-mer subsequences. An operation (for example, a product operation) is performed on the first vectors of the M amino acid k-mer subsequences by using a second mapping matrix to obtain second vectors of the M amino acid k-mer subsequences, and the second vectors of the M amino acid k-mer subsequences compose a first protein vector sequence.

For example, the first vector may be a One-Hot vector, and the second vector may be an Embedding vector. For the protein sequence MTAQDDSYS to be predicted, it may include three amino acid 3-mer subsequences of MTA, QDD and SYS. One-Hot encoding may be performed on each amino acid 3-mer subsequence to obtain the amino acid 3-mer One-Hot vectors, which are V₁^{P}, V₂^{P} and V₃^{P}, respectively. Since the amino acid 3-mer One-Hot vector is an 8000-dimensional sparse vector, the amino acid 3-mer One-Hot vector may be mapped to a dense Embedding vector through a second mapping matrix W₂. That is, according to the following:

Eⱼ^{P}=W₂×Vⱼ^{P} (2)

the j-th amino acid 3-mer Embedding vector Eⱼ^{P} in the protein sequence to be predicted is obtained, where Vⱼ^{P} represents the j-th amino acid 3-mer One-Hot vector in the protein sequence to be predicted, and the second mapping matrix W₂ is a B*8000 parameter matrix. For example, B may be 256 or 128, and the value of B is not specifically limited in the present disclosure. Based on this, the 3-mer Embedding vectors corresponding to the three amino acid 3-mer subsequences can be obtained in sequence, which are E₁^{P}, E₂^{P} and E₃^{P}, respectively, and then the three amino acid 3-mer Embedding vectors compos a first protein vector sequence.

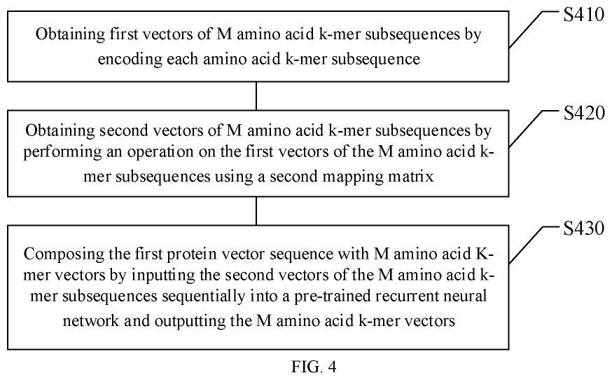

In an example embodiment, after the protein sequence to be predicted is converted into M amino acid k-mer subsequences, referring to FIG. 4, each amino acid k-mer subsequence may be encoded according to steps S410 to S430 to obtain a first protein vector sequence.

In step S410, first vectors of M amino acid k-mer subsequences are obtained by encoding each amino acid k-mer subsequence.

For example, the first vector may be a One-Hot vector. For the protein sequence MTAQDDSYS to be predicted, it may include three amino acid 3-mer subsequences of MTA, QDD and SYS. One-Hot encoding may be performed on each amino acid 3-mer subsequence to obtain the amino acid 3-mer One-Hot vectors, which are V₁^{P}, V₂^{P}, and V₃^{P}, respectively.

In step S420, second vectors of the M amino acid k-mer subsequences are obtained by performing an operation on the first vectors of the M amino acid k-mer subsequences using a second mapping matrix.

The second vector may be an Embedding vector. Since the amino acid 3-mer One-Hot vector is an 8000-dimensional sparse vector, the amino acid 3-mer One-Hot vector may be mapped to a dense Embedding vector through the second mapping matrix W₂ to obtain three amino acid 3-mer embedding vectors, which are E₁^{P}, E₂^{P} and E₃^{P}, respectively.

In step S430, the second vectors of the M amino acid k-mer subsequences are sequentially input into a pre-trained recurrent neural network, M amino acid k-mer vectors are output, and the M amino acid k-mer vectors compose the first protein vector sequence.

In this example, all amino acid 3-mer Embedding vectors in the protein sequence to be predicted can be regarded as a time sequence sequence, so that an operation may be performed on each amino acid 3-mer embedding vector by using a recurrent neural network. For example, after all amino acid 3-mer Embedding vectors (E₁^{P}, E₂^{P}, and E₃^{P}) in the protein sequence MTAQDDSYS to be predicted are obtained, the three amino acid 3-mer Embedding vectors can be sequentially input into the trained LSTM network, and each corresponding amino acid 3-mer vector is output, which is h₁^{P}, h₂^{P}and h₃^{P}, respectively. The three amino acid 3-mer vectors compose a first protein vector sequence.

Specifically, the Embedding vector E₁^{P} corresponding to “MTA” can be firstly input into the LSTM network. Hidden feature extraction can be performed on E₁^{P} through the LSTM network, and a hidden vector h₁^{P} at that moment, such as moment t, can be output. Then, the hidden vector h₁^{P} at moment t can be spliced with the Embedding vector E₂^{P} corresponding to “QDD” at moment t+1, the spliced vector is input into the LSTM network, hidden feature extraction is performed on the spliced vector, and the hidden vector h₂^{P} at moment t+1 is output. Finally, the Embedding vector E₃^{P} corresponding to “SYS” can be input into the LSTM network, the hidden vector h₂^{P} at moment t+1 can be spliced with the Embedding vector E₃^{P}. Hidden feature extraction is performed on the spliced vector to output the hidden vector h₃^{P} at the last moment. In other examples, the operation can be performed on each amino acid 3-mer Embedding vector by using a GRU network. Each amino acid 3-mer One-Hot vector in the protein sequence to be predicted can also be directly input into the GRU network to obtain a corresponding base 3-mer vector, which is not specifically limited in the present disclosure.

In this embodiment, when processing the plurality of amino acid 3-mer Embedding vectors in the protein sequence to be predicted using the LSTM network, the dependency relationship between various amino acid 3-mer Embedding vectors can be learned and memorized. Based on this, the relevance information between the RNA sequence and the protein sequence can be obtained more accurately, and the probability value of interaction between the RNA sequence and the protein sequence is accurately predicted by fusing the relevance information. In addition, it can be understood that, in order to facilitate predicting the probability value of interaction between the RNA and the protein, the dimensions of the base 3-mer vector hᵢ^{R} and the dimensions of the amino acid 3-mer vector hⱼ^{P} output by the LSTM network may be consistent with each other, for example, both of them may be 64 dimensions or 128 dimensions, which is not specifically limited in the present disclosure.

In step S240, a relevance vector sequence of the first RNA vector sequence and a relevance vector sequence of the first protein vector sequence are obtained through a selective attention mechanism model.

In an example embodiment of the present disclosure, the relevance vector sequence of the first RNA vector sequence and the relevance vector sequence of the first protein vector sequence may be obtained through the selective attention mechanism model. The relevance vector sequence of the first RNA vector sequence and/or the relevance vector sequence of the first protein vector sequence are introduced when the probability value of interaction between the input RNA sequence and the protein sequence is predicted, to improve the accuracy of interaction prediction between the RNA and the protein. Among them, the selective attention mechanism model is a machine learning model, in which human visual attention behaviors can be simulated. When humans observe an image, after global scanning of the image, a target region needing attention can be obtained, and then more attention is paid, so that detail information of the target region is obtained. Therefore, human attention behaviors can be abstracted and applied to a machine learning model.

In the selective attention mechanism model, each input vector needs three vector representations, which are query representation query, key representation key and value representation value, respectively. Mapping from query and a series of key-value pairs to output output can be realized through the attention mechanism. The output output can be obtained by performing weighted summation on value, and the weight corresponding to each value can be calculated by query and key through a compatibility function. For example, the compatibility function may be a Softmax function.

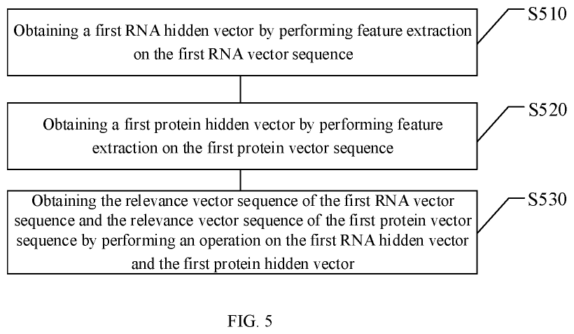

For example, referring to FIG. 5, a relevance vector sequence of the first RNA vector sequence and a relevance vector sequence of the first protein vector sequence may be obtained through a selective attention mechanism model according to steps S510 to S530.

In step S510, a first RNA hidden vector is obtained by performing feature extraction on the first RNA vector sequence.

When the RNA sequence to be predicted is converted into N base k-mer subsequences, the corresponding first RNA vector sequence may be {hᵢ^{R}, i=1,2, . . . , N}. The first RNA vector sequence includes N base k-mer vectors. Before feature extraction is performed on the first RNA vector sequence, three weight matrices may be initialized firstly, which are a query weight matrix, a key weight matrix and a value weight matrix, respectively, so that a query representation, a key representation, and a value representation of each base k-mer vector in the first RNA vector sequence may be obtained according to the three weight matrices. Correspondingly, the first RNA hidden vector obtained by performing feature extraction on each base k-mer vector in the first RNA vector sequence may include a first RNA vector, a second RNA vector, and a third RNA vector. For example, the first query weight matrix may be used to perform an operation on the first RNA vector sequence to obtain the first RNA vector of each base k-mer subsequence. The first key weight matrix may be used to perform an operation on the first RNA vector sequence to obtain the second RNA vector of each base k-mer subsequence. The first value weight matrix may be used to perform an operation on the first RNA vector sequence to obtain the third RNA vector of each base k-mer subsequence. It may be understood that the first RNA vector, the second RNA vector, and the third RNA vector respectively correspond to a query representation, a key representation, and a value representation of the base k-mer vector.

For example, for the i-th base k-mer vector hᵢ^{R} in the first RNA vector sequence, the first query weight matrix W_{q}^{R}, the first key weight matrix Wₖ^{R}, and the first value weight matrix Wᵥ^{R} may be used to perform an operation to obtain the query representation queryRᵢ, the key representation keyRᵢ, and the value representation valueRᵢ of the i-th base k-mer vector, that is:

queryRᵢ=W_{q}^{R}×hᵢ^{R}

keyRᵢ=Wₖ^{R}×hᵢ^{R}

valueRᵢ=Wᵥ^{R}×hᵢ^{R} (3).

The first query weight matrix W_{q}^{R}, the first key weight matrix Wₖ^{R}, the first value weight matrix Wᵥ^{R} may be used to sequentially perform an operation on each base k-mer vector in the first RNA vector sequence, or to perform an operation on all base k-mer vectors in the first RNA vector sequence at the same time to improve the interaction prediction efficiency, which is not specifically limited in the present disclosure. It should be noted that the first RNA hidden vector obtained by performing feature extraction on the first RNA vector sequence may include hidden vectors of a plurality of base k-mer subsequences, and the hidden vector of each base k-mer subsequence includes a first RNA vector, a second RNA vector, and a third RNA vector. For example, the hidden vector of the i-th base k-mer subsequence may include a first RNA vector queryRᵢ, a second RNA vector keyRᵢ, and a third RNA vector valueR.

In step S520, a first protein hidden vector is obtained by performing feature extraction on the first protein vector sequence.

Similarly, when the protein sequence to be predicted is converted into M amino acid k-mer subsequences, the corresponding first protein vector sequence may be {hⱼ^{P}, j=1,2, . . . , M }. The protein vector sequence may include M amino acid k-mer vectors. When performing feature extraction on the first protein vector sequence through the selective attention mechanism model, a query representation, a key representation, and a value representation of each amino acid k-mer vector in the first protein vector sequence may also be obtained according to the query weight matrix, the key weight matrix, and the value weight matrix.

Correspondingly, the first protein hidden vector obtained by performing feature extraction on each amino acid k-mer vector in the first protein vector sequence may include a first protein vector, a second protein vector, and a third protein vector. For example, a second query weight matrix can be used to perform an operation on the first protein vector sequence to obtain the first protein vector of each amino acid k-mer subsequence. A second key weight matrix can be used to perform an operation on the first protein vector sequence to obtain the second protein vector of each amino acid k-mer subsequence. A second value weight matrix can be used to perform an operation on the first protein vector sequence to obtain the third protein vector of each amino acid k-mer subsequence. It can be understood that the first protein vector, the second protein vector, and the third protein vector respectively correspond to a query representation, a key representation, and a value representation of the amino acid k-mer vector.

For example, for the j-th amino acid k-mer vector hⱼ^{p} in the first protein vector sequence, the second query weight matrix W_{q}^{p}, the second key weight matrix Wₖ^{p}, and the second value weight matrix Wᵥ^{p} may be used to perform an operation to obtain the query representation queryPⱼ, the key representation keyPⱼ, and the value representation valuePⱼ of the j-th amino acid k-mer vector, that is,

queryPⱼ=W_{q}^{p}×hⱼ^{p}

keyPⱼ=Wₖ^{p}×hⱼ^{p}

valuePⱼ=Wᵥ^{p}×hⱼ^{p} (4).

The second query weight matrix W_{q}^{p}, the second key weight matrix Wₖ^{p}, and the second value weight matrix Wᵥ^{p} may be used to sequentially perform an operation on each amino acid k-mer vector in the first protein vector sequence, or to perform an operation on all amino acid k-mer vectors in the first protein vector sequence at the same time to improve the interaction prediction efficiency, which is not specifically limited in the present disclosure. It should be noted that the first protein hidden vector obtained by performing feature extraction on the first protein vector sequence may include hidden vectors of a plurality of amino acid k-mer subsequences, and the hidden vector of each amino acid k-mer subsequence includes a first protein vector, a second protein vector, and a third protein vector. For example, the hidden vector of the j-th amino acid k-mer subsequence may include a first protein vector queryPⱼ, a second protein vector keyPⱼ, and a third protein vector valuePⱼ.

In step S530, a relevance vector sequence of the first RNA vector sequence and a relevance vector sequence of the first protein vector sequence are obtained by performing an operation on the first RNA hidden vector and the first protein hidden vector.

After obtaining the first RNA hidden vector of each base k-mer subsequence and the first protein hidden vector of each amino acid k-mer subsequence, an operation can be performed on the first RNA hidden vector of each base k-mer subsequence and the first protein hidden vector of each amino acid k-mer subsequence to obtain a relevance vector sequence of the first RNA vector sequence and a relevance vector sequence of the first protein vector sequence. Among them, the relevance vector sequence of the first protein vector sequence can be fused into the first protein vector sequence, and at the same time, the relevance vector sequence of the first RNA vector sequence can also be fused into the first RNA vector sequence, so as to introduce more sequence information to predict the probability value of interaction between the RNA and the protein, thus improving the prediction accuracy. It can be understood that only the relevance vector sequence of the first protein vector sequence can be fused into the first protein vector sequence, so as to predict the probability value of interaction using the first protein vector sequence fused with the RNA sequence information and the first RNA vector sequence. Only the relevance vector sequence of the first RNA vector sequence can also be fused into the first RNA vector sequence, so as to predict the probability value of interaction using the first RNA vector sequence fused with the protein sequence information and the first RNA vector sequence. The relevance vector sequence of the first protein vector sequence and the relevance vector sequence of the first RNA vector sequence can also be directly used to predict the probability value of interaction, which is not specifically limited in the present disclosure.

For example, first RNA hidden vectors of the i-th base k-mer subsequence are a first RNA vector (queryRᵢ), a second RNA vector (keyRᵢ) and a third RNA vector (valueRᵢ), respectively. The first protein hidden vectors of the j-th amino acid k-mer subsequence are a first protein vector (queryPⱼ), a second protein vector (keyPⱼ), and a third protein vector (valuePⱼ), respectively. A similarity between the first RNA vector and the second protein vector may be calculated to obtain a first RNA attention score (i.e., a weight), and the third protein vector may be summed according to the first RNA attention score to obtain a relevance vector sequence of the first RNA vector sequence. For example, an attention scoring function can be used to calculate the similarity between the first RNA vector and the second protein vector, and the calculated similarity value may be normalized. The normalized result is the first RNA attention score. Among them, the used attention scoring function may be a dot product operation. The attention score may also be calculated by using an additive model, a zoom click model, a bilinear model, etc., which is not specifically limited in the present disclosure. Specifically, according to the following:

$${a}_{i , j}^{R} = \frac{{e}^{〈 {queryR}_{i} , {keyP}_{j} 〉}}{{\sum }_{k = 1}^{k = M} {e}^{〈 {queryR}_{i} , {keyP}_{k} 〉}} , \qquad ( 5 )$$

the first RNA attention score a_{i, j}^{R} may be obtained through normalization. In some embodiments, a Softmax function may be used to perform normalization. Among them, [symbol] queryRᵢ, keyPⱼ [symbol] represents performing a dot product operation on the i-th first RNA vector queryRᵢ and the j-th second protein vector keyPⱼ. The value of k may be [1, M], M is the number of amino acid k-mer subsequences included in the protein to be predicted, and [symbol] queryRᵢ, keyPₖ [symbol] represents performing a dot product operation on the i-th first RNA vector queryRᵢ and the k-th second protein vector keyPₖ. During normalization, Σₖ₌₁^{k=M} [symbol] represents a sum of the dot product results of the i-th first RNA vector and all second protein vectors, and [symbol] represents a dot product result of the i-th first RNA vector queryRᵢ and the j-th second protein vector keyPⱼ under the index e, so as to widen the difference of each dot product result by using the index e. In addition, in this example, two different types of information of the RNA sequence and the protein sequence are fused, so that the index can be used in formula (5) for smoothing, so as to more accurately obtain the relevance information between the RNA sequence and the protein sequence. After obtaining the first RNA attention score, according to the following:

vᵢ^{P}=Σₖ₌₁^{k=M}a_{i, k}^{R}×valuePₖ (6),

weighted summation may be performed on all third protein vectors to obtain a relevance vector vᵢ^{P} of the i-th first RNA vector sequence, where the first RNA attention score a_{i, k}^{R} represents the weight corresponding to the k-th third protein vector valuePₖ.

Similarly, a similarity between the first protein vector and the second RNA vector can also be calculated to obtain a first protein attention score (i.e., a weight). The third RNA vector is summed according to the first protein attention score to obtain a relevance vector sequence of the first protein vector sequence. For example, an attention scoring function can be used to calculate the similarity between the first protein vector and the second RNA vector. The calculated similarity value is normalized, and the normalized result is the first protein attention score. Among them, the used attention scoring function may be a dot product operation. The attention score can also be calculated by using an additive model, a zoom click model, a bilinear model, etc., which is not specifically limited in the present disclosure. Specifically, according to the following:

$${a}_{j , i}^{P} = \frac{{e}^{〈 que {ryP}_{j} , {keyR}_{i} 〉}}{{\sum }_{k = 1}^{k = N} {e}^{〈 {queryP}_{j} , {keyR}_{k} 〉}} , \qquad ( 7 )$$

the first protein attention score a_{j, i}^{P} may be obtained through normalization. In some embodiments, a Softmax function may be used to perform normalization. Among them, [symbol] queryPⱼ, keyRᵢ [symbol] represents performing a dot product operation on the j-th first protein vector queryPⱼ and the i-th second RNA vector keyRᵢ. The value of k may be [1, N], N is the number of base k-mer subsequences included in the RNA to be predicted, and [symbol] queryPⱼ, keyRₖ [symbol] represents performing a dot product operation on the j-th first protein vector queryPⱼ and the k-th second RNA vector keyRₖ. During normalization, Σₖ₌₁^{k=N} [symbol] represents a sum of the dot product results of the j-th first protein vector and all second RNA vectors, and [symbol] represents a dot product result of the i-th first protein vector queryPⱼ and the j-th second RNA vector keyRⱼ under the index e, so as to widen the difference of each dot product result by using the index e. After obtaining the first protein attention score, according to the following:

vⱼ^{R}=Σₖa_{j, k}^{P}×valueRₖ (8),

weighted summation is performed on all third RNA vectors valueRₖ to obtain a relevance vector vⱼ^{R} of the j-th first protein vector sequence, where the first protein attention score a_{j, k}^{P} represents the weight corresponding to the k-th third RNA vector valueRₖ.

In this example, the relevance information between the RNA sequence and the protein sequence is determined by using a selective attention mechanism model, so as to fuse the RNA sequence, the protein sequence, and the relevance information. When the sequence information obtained by fusion is introduced to predict the probability value of interaction between the RNA sequence and the protein sequence, the accuracy of predicting the probability value of interaction between the RNA and the protein can be improved.

In step S250, the probability value of interaction between the RNA sequence to be predicted and the protein sequence to be predicted is determined according to the relevance vector sequence of the first RNA vector sequence and the relevance vector sequence of the first protein vector sequence.

In an example embodiment of the present disclosure, the probability value of interaction between the RNA sequence to be predicted and the protein sequence to be predicted may be determined according to the relevance vector sequence of the first RNA vector sequence and the relevance vector sequence of the first protein vector sequence. A second RNA vector sequence and a second protein vector sequence may also be obtained according to the relevance vector sequence of the first RNA vector sequence and the relevance vector sequence of the first protein vector sequence. For example, the relevance vector sequence of the first RNA vector sequence may be spliced with the first RNA vector sequence to obtain an RNA fusion vector sequence. The RNA fusion vector sequence is input into a pre-trained recurrent neural network, and a second RNA vector sequence is output. Similarly, the relevance vector sequence of the first protein vector sequence may be spliced with the first protein vector sequence to obtain a protein fusion vector sequence. The spliced vector sequence is input into a pre-trained recurrent neural network, and a second protein vector sequence is output. Finally, the probability value of interaction between the RNA sequence to be predicted and the protein sequence to be predicted may be determined according to the second RNA vector sequence and the second protein vector sequence. It can be understood that the RNA fusion vector sequence and the protein fusion vector sequence may also be used to determine the probability value of interaction between the RNA sequence to be predicted and the protein sequence to be predicted, which is not limited in the present disclosure.

For example, for the i-th base k-mer vector hᵢ^{R} in the first RNA vector sequence, the relevance vector of the base k-mer vector is vᵢ^{P}. A dot product operation can be performed on the i-th base k-mer vector hᵢ^{R} and the relevance vector vᵢ^{P} of the base k-mer vector to splice them, that is [symbol] hᵢ^{R}, vᵢ^{P} [symbol] . The operation result is the RNA vector sequence fused with the protein sequence information, which may be recorded as hᵢ^{R1}. For the j-th amino acid k-mer vector hⱼ^{P} in the first protein vector sequence, the relevance vector of the amino acid k-mer vector is vⱼ^{R}. A dot product operation may also be performed on the j-th amino acid k-mer vector hⱼ^{P} and the relevance vector vⱼ^{R} of the amino acid k-mer vector to splice them, that is [symbol] hⱼ^{P}, vⱼ^{R} [symbol] . The operation result is the protein vector sequence fused with the RNA sequence information, which may be recorded as hⱼ^{P1}.

Taking the RNA sequence to be predicted as an example, in order to further capture the relevance relationship between the various base k-mer vectors in the sequence {hᵢ^{R1}, i=1,2, . . . , N} after the protein sequence information is fused, the sequence {hᵢ^{R1}, i=1,2, . . . , N} may be used as an input of the LSTM network, and hidden feature extraction can be performed sequentially on hᵢ^{R1} through the LSTM network. In an example embodiment of the present disclosure, the output of the LSTM network at the last moment may be used as the final vector representation of the RNA sequence to be predicted, or the outputs of the LSTM network at all moments may also be averaged to serve as the final vector representation of the RNA sequence to be predicted. For example, the hidden vector h_{N}^{R2} output at the last moment may be used as a second RNA vector sequence. Similarly, when {hⱼ^{P1}, j=1,2, . . . , M} is used as the input of the LSTM network, the output of the LSTM network at the last moment can be used as the final vector representation of the protein sequence to be predicted, or the outputs of the LSTM network at all moments can also be averaged to serve as the final vector representation of the protein sequence to be predicted. For example, the hidden vector h_{M}^{P2} output at the last moment may be used as a second protein vector sequence. In other examples, hidden feature extraction may also be performed on hᵢ^{R1}/hⱼ^{P1} through the GRU network, which is not specifically limited in the present disclosure.

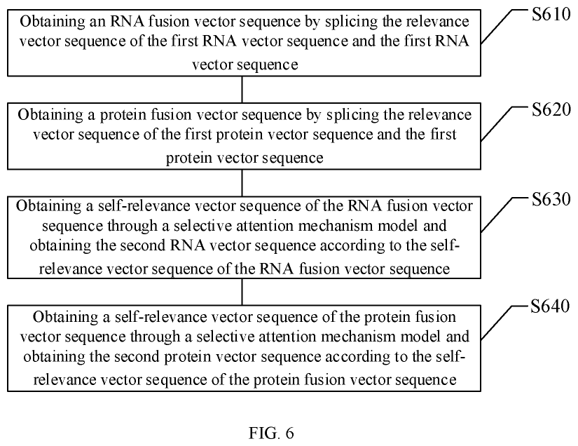

In an example embodiment, referring to FIG. 6, a second RNA vector sequence and a second protein vector sequence may also be obtained by performing information fusion through a selective attention mechanism model according to steps S610 to S640.

In step S610, an RNA fusion vector sequence is obtained by splicing the relevance vector sequence of the first RNA vector sequence and the first RNA vector sequence. For example, for the i-th base k-mer vector hᵢ^{R} in the first RNA vector sequence, the relevance vector of the base k-mer vector is vᵢ^{P}. A dot product operation may be performed on the i-th base k-mer vector hᵢ^{R} and the relevance vector vᵢ^{P} of the base k-mer vector to splice them, that is [symbol] hᵢ^{R}, vᵢ^{P} [symbol] , so as to obtain an RNA fusion vector sequence {hᵢ^{R1}=1,2, . . . , N}.

Then, the RNA fusion vector sequence may be used as the input of the LSTM network, and hidden feature extraction may be performed through the LSTM network to capture the relevance relationship between the various base k-mer vectors of the RNA fusion vector sequence. The outputs of the LSTM network at all moments may be obtained, which are recorded as {hᵢ^{R2}, i=1,2, . . . , N}.

In step S620, a protein fusion vector sequence is obtained by splicing the relevance vector sequence of the first protein vector sequence and the first protein vector sequence.

For example, for the j-th amino acid k-mer vector hⱼ^{P} in the first protein vector sequence, the relevance vector of the amino acid k-mer vector is vⱼ^{R}. A dot product operation may also be performed on the j-th amino acid k-mer vector hⱼ^{P} and the relevance vector vⱼ^{R} of the amino acid k-mer vector to splice them, that is [symbol] hⱼ^{P}, vⱼ^{R} [symbol] , so as to obtain a protein fusion vector sequence {hⱼ^{P1}, j=1,2, . . . , M}.

The protein fusion vector sequence can be used as the input of the LSTM network, and hidden feature extraction may be performed through the LSTM network to capture the relevance relationship between the various amino acid k-mer vectors of the protein fusion vector sequence. The outputs of the LSTM network at all moments may be obtained, which are recorded as {hⱼ^{P2}, j=1,2, . . . , M}.

In step S630, a self-relevance vector sequence of the RNA fusion vector sequence is obtained through a selective attention mechanism model, and the second RNA vector sequence is obtained according to the self-relevance vector sequence of the RNA fusion vector sequence.

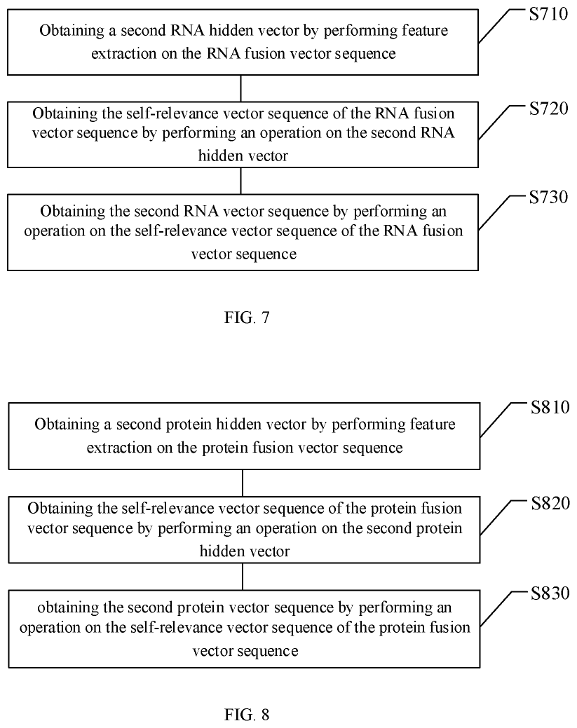

After a new RNA fusion vector sequence {hᵢ^{R2}, i=1,2, . . . , N} is obtained, in order to obtain the relevance information between the various base k-mer vectors in the RNA fusion vector sequence, a selective attention mechanism model may be used to obtain an self-relevance vector sequence of the RNA fusion vector sequence. Referring to FIG. 7, step S630 may further include step S710 to step S730.

In step S710, a second RNA hidden vector is obtained by performing feature extraction on the RNA fusion vector sequence.

Among them, the second RNA hidden vector obtained by performing feature extraction on each base k-mer vector in the RNA fusion vector sequence may include a fourth RNA vector, a fifth RNA vector, and a sixth RNA vector. For example, a third query weight matrix may be used to perform operation on the RNA fusion vector sequence to obtain the fourth RNA vector of each base k-mer subsequence. A third key weight matrix may be used to perform operation on the RNA fusion vector sequence to obtain the fifth RNA vector of each base k-mer subsequence. A third value weight matrix may be used to perform operation on the RNA fusion vector sequence to obtain the sixth RNA vector of each base k-mer subsequence.

For example, after inputting the RNA fusion vector sequence into the LSTM network, the obtained RNA fusion vector sequence is changed into {hᵢ^{R2}, i=1,2, . . . , N}. For the i-th base k-mer vector hᵢ^{R2}, a third query weight matrix W_{q}^{R2}, a third key weight matrix Wₖ^{R2} and the third value weight matrix Wᵥ^{R2} may be used to perform operation to obtain the query representation queryRᵢ², the key representation keyRᵢ² and the value representation valueRᵢ² of the i-th base k-mer vector hᵢ^{R2}, that is:

queryRᵢ²=W_{q}^{R2}×hᵢ^{R2}

keyRᵢ²=Wₖ^{R2}×hᵢ^{R2}

valueRᵢ²=Wᵥ^{R2}×hᵢ^{R2} (9).

The third query weight matrix W_{q}^{R2}, the third key weight matrix Wₖ^{R2}, and the third value weight matrix Wᵥ^{R2} can be used to sequentially perform operation on each base k-mer vector hᵢ^{R2} in the RNA fusion vector sequence, or to perform operation on all base k-mer vectors in the RNA fusion vector sequence at the same time to improve the interaction prediction efficiency, which is not specifically limited in the present disclosure.

In step S720, a self-relevance vector sequence of the RNA fusion vector sequence is obtained by performing an operation on the second RNA hidden vector;

After obtaining the second RNA hidden vector of each base k-mer subsequence, a self-relevance vector sequence of the RNA fusion vector sequence can be obtained by performing operation on the fourth RNA vector (queryRᵢ²), the fifth RNA vector (keyRᵢ²) and the sixth RNA vector (valueRᵢ^{R2}). For example, a similarity between the fourth RNA vector and the fifth RNA vector may be calculated to obtain a second RNA attention score. The sixth RNA vector may be summed according to the second RNA attention score to obtain the self-relevance vector sequence of the RNA fusion vector sequence. That is, the relevance information between the various base k-mer vectors in the RNA fusion vector sequence is obtained. Specifically, according to the following:

$${a}_{i , j}^{R 2} = \frac{〈 {queryR}_{i}^{2} , {keyR}_{j}^{2} 〉}{{\sum }_{k = 1}^{k = N} 〈 {queryR}_{i}^{2} , {keyR}_{k}^{2} 〉} , \qquad ( 10 )$$

the second RNA attention score may be obtained through normalization, where [symbol] queryRᵢ², keyRⱼ² [symbol] represents performing a dot product operation on the i-th fourth RNA vector queryRᵢ² and the j-th fifth RNA vector keyRⱼ², and Σₖ₌₁^{k=N} [symbol] queryRᵢ², keyRₖ² [symbol] represents a sum of the dot product results of the i-th fourth RNA vector and all fifth RNA vectors. It can be seen that the attention scoring function used in formula (10) is a dot product operation. An additive model, a zoom click model, a bilinear model, etc., may also be used to calculate the attention score, which is not specifically limited in the present disclosure. In addition, in this example, the same type of information of the RNA sequence and the RNA sequence is fused, so that the relevance information between the various base k-mer vectors can be accurately obtained without performing smoothing using the index in the formula (10).

hᵢ^{R3}=Σⱼ₌₁^{j=N} a_{i, j}^{R2}×valueRⱼ² (11),

weighted summation may be performed on all sixth RNA vectors to obtain a self-relevance vector hᵢ^{R3} of the i-th base k-mer vector hᵢ^{R2} in the RNA fusion vector sequence, that is, the base k-mer vectors fused with other base information.

In step S730, the second RNA vector sequence is obtained by performing an operation on the self-relevance vector sequence of the RNA fusion vector sequence.

According to the following:

$${v}_{RNA} = \frac{{\sum }_{i = 1}^{i = N} {h}_{i}^{R 3}}{N} , \qquad ( 12 )$$

the second RNA vector sequence may be calculated and obtained, which is recorded as v_{RNA}. Among them, hᵢ^{R3} is the i-th base k-mer vector obtained after other base information is fused. When v_{RNA} obtained from average operation is used to represent a complete RNA sequence to be predicted, accurate interaction prediction may be performed without considering whether the sequence length of the RNA sequence to be predicted and the sequence length of the protein sequence are consistent with each other. It may be understood that the summation result of Σᵢhᵢ^{R3} may also be used as a second RNA vector sequence, and {hᵢ^{R3}, i=1,2, . . . , N} may also be directly used as a second RNA vector sequence, which is not specifically limited in the present disclosure.

It should be noted that, in some embodiments, after obtaining the RNA fusion vector sequence and the protein fusion vector sequence, the step of inputting the RNA fusion vector sequence and the protein fusion vector sequence into the LSTM network can be omitted. The selective attention mechanism model is directly used to obtain the self-relevance vector sequence of the RNA fusion vector sequence and the self-relevance vector sequence of the protein fusion vector sequence, and the interaction prediction is performed by using the self-relevance vector sequence of the RNA fusion vector sequence and the self-relevance vector sequence of the protein fusion vector sequence.

In step S640, a self-relevance vector sequence of the protein fusion vector sequence is obtained through a selective attention mechanism model, and a second protein vector sequence is obtained according to the self-relevance vector sequence of the protein fusion vector sequence.

After a new protein fusion vector sequence {hⱼ^{P2}, j=1,2, . . . , M} is obtained, in order to obtain the relevance information between the various amino acid k-mer vectors in the protein fusion vector sequence, a selective attention mechanism model may be used to obtain a self-relevance vector sequence of the protein fusion vector sequence. Referring to FIG. 8, step S640 may further include step S810 to step S830.

In step S810, a second protein hidden vector is obtained by performing feature extraction on the protein fusion vector sequence.

Among them, the second protein hidden vector obtained by performing feature extraction on each amino acid k-mer vector in the protein fusion vector sequence may include a fourth protein vector, a fifth protein vector, and a sixth protein vector. For example, a fourth query weight matrix may be used to perform operation on the protein fusion vector sequence to obtain the fourth protein vector of each amino acid k-mer subsequence. A fourth key weight matrix may be used to perform operation on the protein fusion vector sequence to obtain the fifth protein vector of each amino acid k-mer subsequence. A fourth value weight matrix may be used to perform operation on the protein fusion vector sequence to obtain the sixth protein vector of each amino acid k-mer subsequence.

For example, after inputting the protein fusion vector sequence into the LSTM network, the obtained protein fusion vector sequence is changed into {hⱼ^{P2}, j=1,2, . . . , N}. For the j-th base k-mer vector, a fourth query weight matrix W_{q}^{P2}, a fourth key weight matrix Wₖ^{P2}, and a fourth value weight matrix Wᵥ^{P2} may be used to perform operation to obtain the query representation queryPⱼ², the key representation keyPⱼ² and the value representation valuePⱼ² of the j-th base k-mer vector hⱼ^{P2}, that is:

queryPⱼ²=W_{q}^{P2}×hⱼ^{P2}

keyPⱼ²=Wₖ^{P2}×hⱼ^{P2}

valuePⱼ²=Wᵥ^{P2}×hⱼ^{P2} (13).

The third query weight matrix W_{q}^{R2}, the third key weight matrix Wₖ^{R2}, and the third value weight matrix Wᵥ^{R2} can be used to sequentially perform operation on each base k-mer vector hᵢ^{R2} in the RNA fusion vector sequence, or to perform operation on all base k-mer vectors in the RNA fusion vector sequence at the same time to improve the interaction prediction efficiency, which is not specifically limited in the present disclosure.

In step S820, a self-relevance vector sequence of the protein fusion vector sequence is to obtained by performing an operation on the second protein hidden vector.

After the second protein hidden vector of each amino acid k-mer subsequence is obtained, a self-relevance vector sequence of the protein fusion vector sequence may be obtained by performing operation on the fourth protein vector (queryPⱼ²), the fifth protein vector (keyPⱼ²) and the sixth protein vector (valuePⱼ²). For example, a similarity between the fourth protein vector and the fifth protein vector can be calculated to obtain the second protein attention score. The sixth protein vector is summed according to the second protein attention score to obtain the self-relevance vector sequence of the protein fusion vector sequence. That is, the relevance information between the various amino acid k-mer vectors in the protein fusion vector sequence is obtained.

Specifically, according to the following:

$${a}_{j , i}^{P 2} = \frac{〈 {queryP}_{j}^{2} , {keyP}_{i}^{2} 〉}{{\sum }_{k = 1}^{k = M} 〈 {queryP}_{j}^{2} , {keyP}_{k}^{2} 〉} , \qquad ( 14 )$$

the second protein attention score may be obtained through normalization, where [symbol] queryPⱼ², keyPᵢ² [symbol] represents performing a dot product operation on the j-th fourth protein vector queryRᵢ² and the i-th fifth protein vector keyRⱼ², and Σₖ₌₁^{k=M} [symbol] queryPⱼ², keyPₖ² [symbol] represents a sum of the dot product results of the j-th fourth protein vector and all fifth protein vectors. After obtaining the second protein attention score according to the following:

hⱼ^{P3}=Σᵢ₌₁^{i=M} a_{j, i}^{P2}×valuePᵢ² (15),

weighted summation may be performed on all sixth protein vectors to obtain a self-relevance vector hⱼ^{R3} of the j-th amino acid k-mer vector hⱼ^{P2} in the protein fusion vector sequence, that is, the amino acid k-mer vector fused with other amino acid information.

In step S830, the second protein vector sequence is obtained by performing an operation on the self-relevance vector sequence of the protein fusion vector sequence According to the following:

$${v}_{P} = \frac{{\sum }_{j = 1}^{j = M} {h}_{j}^{P 3}}{M} , \qquad ( 16 )$$

the second protein vector sequence may be calculated and obtained, which is recorded as v_{P}. Among them, hⱼ^{R3} is the j-th amino acid k-mer vector obtained after other amino acid information is fused. When v_{P} obtained from average operation is used to represent a complete protein sequence to be predicted, accurate interaction prediction may be performed without considering whether the sequence length of the protein sequence to be predicted and the sequence length of the RNA sequence are consistent with each other. It may be understood that the summation result of Σⱼhⱼ^{P3} may also be directly used as a second protein vector sequence, and {hⱼ^{P3}, j=1,2, . . . , M} may also be directly used as a second protein vector sequence, which is not specifically limited in the present disclosure.

In an example embodiment of the present disclosure, the probability value of interaction between the RNA sequence to be predicted and the protein sequence to be predicted needs to be predicted, and the obtained prediction result may be that there is an interaction between the RNA sequence to be predicted and the protein sequence to be predicted, or may be that there is no interaction between the RNA sequence to be predicted and the protein sequence to be predicted, that is, performing binary classification prediction.

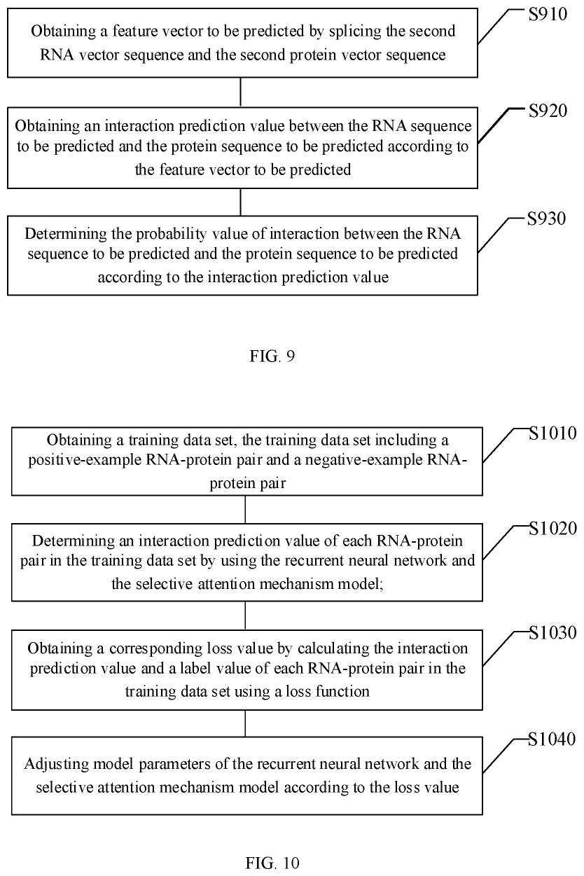

After obtaining the second RNA vector sequence and the second protein vector sequence, referring to FIG. 9, the probability value of interaction between the RNA sequence to be predicted and the protein sequence to be predicted may be determined according to steps S910 to S930.

In step S910, a feature vector to be predicted is obtained by splicing the second RNA vector sequence and the second protein vector sequence.

For example, when the obtained second RNA vector sequence is v_{RNA}, and the obtained second protein vector sequence is V_{P}, a dot product operation may be performed on the obtained second RNA vector sequence v_{RNA} and the obtained second protein vector sequence v_{P} to splice them, that is, [symbol] v_{RNA}, v_{P} [symbol] and the original feature vector obtained by splicing may be recorded as v. In other examples, if the obtained second RNA vector sequence is h_{N}^{R2} and the obtained second protein vector sequence is h_{M}^{P2}, a dot product operation may be performed on the obtained second RNA vector sequence h_{N}^{R2} and the obtained second protein vector sequence h_{M}^{P2} to splice them, that is [symbol] h_{N}^{R2}, h_{M}^{P2} [symbol] , and the probability value of interaction between the RNA sequence and the protein sequence may also be predicted according to the original feature vector obtained by splicing, which is not specifically limited in the present disclosure.

In order to facilitate performing subsequent binary classification prediction, the original feature vector v may be mapped to a two-dimensional feature vector to be predicted through a third mapping matrix W₃. That is, according to:

c=W₃×v (17)

a feature vector c to be predicted may be obtained, where c is a 2-dimensional feature vector [c₀, c₁] to be predicted, the third mapping matrix W₃ is a 2*C parameter matrix, v is the original feature vector, and a value of C is consistent with a dimension of the original feature vector.

In step S920, an interaction prediction value between the RNA sequence to be predicted and the protein sequence to be predicted is obtained according to the feature vector to be predicted.

After obtaining the feature vector to be predicted for performing interaction prediction, an interaction prediction value between the RNA sequence to be predicted and the protein sequence to be predicted can be obtained according to the feature vector to be predicted, so as to determine the probability value of interaction between the RNA sequence to be predicted and the protein sequence to be predicted according to the interaction prediction value.

For example, the feature vector to be predicted may be input into a classifier, and the probability value of interaction between the RNA sequence to be predicted and the protein sequence to be predicted is classified according to the feature vector to be predicted. After the classification is completed, an interaction prediction value between the RNA sequence to be predicted and the protein sequence to be predicted is output. For example, the probability value of interaction between the RNA sequence to be predicted and the protein sequence to be predicted may be predicted using a Softmax classifier. Specifically, the feature vector to be predicted may be converted by using a Softmax classifier to obtain that the probability value of interaction between the RNA sequence to be predicted and the protein sequence to be predicted belongs to the probability distribution of “presence of interaction” and the probability distribution of “absence of interaction”, respectively.

For example, the probability value of presence of interaction between the RNA sequence to be predicted and the protein sequence to be predicted obtained through a Softmax classifier is as following:

$$P ( 1 \vert r , p ) = \frac{{e}^{{c}_{1}}}{{e}^{{c}_{1}} + {e}^{{c}_{0}}} . \qquad ( 18 )$$

The obtained probability value of absence of interaction between the RNA sequence to be predicted and the protein sequence to be predicted is as following:

$$P ( 0 \vert r , p ) = \frac{{e}^{{c}_{0}}}{{e}^{{c}_{1}} + {e}^{{c}_{0}}} . \qquad ( 19 )$$

Among them, r represents an RNA sequence to be predicted, p represents a protein sequence to be predicted, c₀ is a first feature value of the feature vector to be predicted, and c₁ represents a second feature value of the feature vector to be predicted. When the feature vector to be predicted is a 2-dimensional vector, the vector is (c₀, c₁). In other examples, a logistic regression classifier, an SVM (Support Vector Machine) classifier may also be used to perform binary classification prediction, so as to obtain an interaction prediction value between the RNA sequence to be predicted and the protein sequence to be predicted according to the feature vector to be predicted, which is not specifically limited in the present disclosure.

In step S930, the probability value of interaction between the RNA sequence to be predicted and the protein sequence to be predicted is determined according to the interaction prediction value.

After obtaining an interaction prediction value between the RNA sequence to be predicted and the protein sequence to be predicted by using a classifier, the probability value of interaction between the RNA sequence to be predicted and the protein sequence to be predicted can be determined according to the interaction prediction value. For example, if the interaction prediction value satisfies a preset threshold condition, it may be determined that there is an interaction between the RNA sequence to be predicted and the protein sequence to be predicted.

For example, the probability value P(1|r, p) of presence of interaction between the RNA sequence to be predicted and the protein sequence to be predicted may be obtained by using a Softmax classifier. Among them, P(1|r, p) may be any value between 0 and 1. For example, the probability threshold for presence of interaction may be preset to be 0.5. When P(1|r, p)>0.5, the prediction result may be marked as 1, that is, it may be determined that there is an interaction between the RNA sequence to be predicted and the protein sequence to be predicted. When P(1|r, p)<0.5, the prediction result may be marked as 0, that is, it may be determined that there is no interaction between the RNA sequence to be predicted and the protein sequence to be predicted. In other examples, it may also be configured that, when P(1|r, p)≥0. 5, it may be determined that there is an interaction between the RNA sequence to be predicted and the protein sequence to be predicted. When P(1|r, p)<0.5, it may be determined that there is no interaction between the RNA sequence to be predicted and the protein sequence to be predicted. Finally, the prediction result of the probability value of interaction between the RNA sequence to be predicted and the protein sequence to be predicted may be output to the terminal device for the user to view. It should be noted that only the probability value of presence of interaction between the RNA sequence to be predicted and the protein sequence to be predicted may be output, or only the probability value of absence of interaction between the RNA sequence to be predicted and the protein sequence to be predicted may be output, or the probability value of presence of interaction and the probability value of absence of interaction between the RNA sequence to be predicted and the protein sequence to be predicted may also be output at the same time, which is not specifically limited in the present disclosure.

In an example embodiment of the present disclosure, when the probability value of interaction between the RNA sequence to be predicted and the protein sequence to be predicted is determined according to the relevance vector sequence of the first RNA vector sequence and the relevance vector sequence of the first protein vector sequence, the relevance vector sequence of the first RNA vector sequence and the relevance vector sequence of the first protein vector sequence can be directly spliced to obtain the feature vector to be predicted. The feature vector to be predicted is input into the classifier to obtain the interaction prediction value between the RNA sequence to be predicted and the protein sequence to be predicted, thus determining the probability value of interaction between the RNA sequence to be predicted and the protein sequence to be predicted. The RNA fusion vector sequence and the protein fusion vector sequence can also be directly spliced to obtain a feature vector to be predicted, and the feature vector to be predicted is input into the classifier to obtain an interaction prediction value between the RNA sequence to be predicted and the protein sequence to be predicted, so as to determine the probability value of interaction between the RNA sequence to be predicted and the protein sequence to be predicted, which is not limited in the present disclosure. In an example embodiment of the present disclosure, referring to FIG. 10, the recurrent neural network and the selective attention mechanism model may be pre-trained according to steps S1010 to S1040, to optimize all model parameters in each prediction model, so that the RNA sequence and the protein sequence which are unknown in probability value of interaction can be predicted according to the final model obtained by training.

In step S1010, a training data set is obtained, where the training data set includes a positive-example RNA-protein pair and a negative-example RNA-protein pair.

For example, each model may be trained based on the RPI1807 data set. There are 3243 RNA-protein pairs in the data set, specifically including 1807 pairs of positive examples and 1436 pairs of negative examples. Among them, the positive example may indicate that there is an interaction between the RNA sequence and the protein sequence in the RNA-protein pair, and the negative example may indicate that there is no interaction between the RNA sequence and the protein sequence in the RNA-protein pair. 1200 positive examples and 1000 negative examples may be selected as the training data set. All RNA-protein pairs may also be selected as the training data set. It can be understood that the number of RNA-protein pairs in the training data set is merely illustrative, and any number of RNA-protein pairs may be obtained to perform a plurality of times of training on each model, so as to improve the performance of each model. It should be noted that the positive-example RNA-protein pair may be marked, and the obtained label value is “1”, that is, it indicates that there is an interaction in the RNA-protein pair. The negative example RNA-protein pair may be marked, and the obtained label value is “0”, that is, it indicates that there is no interaction in the RNA-protein pair. It can be understood that, in other examples, experiments may also be performed based on the RPI2241 data set, the RPI369 data set, etc., which is not specifically limited in the present disclosure.

In step S1020, an interaction prediction value of each RNA-protein pair in the training data set is determined by using the recurrent neural network and a selective attention mechanism model.

Similarly, a corresponding first RNA vector sequence and a corresponding first protein vector sequence may be obtained by using a recurrent neural network to encode the RNA sequence and the protein sequence in each RNA-protein pair in the training data set. A relevance vector sequence of the first RNA vector sequence and a relevance vector sequence of the first protein vector sequence may be obtained by using a selective attention mechanism model. A second RNA vector sequence and a second protein vector sequence may be obtained according to the relevance vector sequence of the first RNA vector sequence and the relevance vector sequence of the first protein vector sequence, and a feature vector to be predicted may be obtained by performing splicing and dimension reduction processing on the second RNA vector sequence and the second protein vector sequence. Finally, an interaction prediction value of each RNA-protein pair may be obtained by using a classifier to perform classification prediction on the feature vector to be predicted.

In step S1030, a corresponding loss value is obtained by calculating an interaction prediction value and a label value of each RNA-protein pair in the training data set using a loss function.

For each RNA-protein pair in the training data set, there is a label value. For example, the label value of each pair of positive examples is 1, and the label value of each pair of negative examples is 0. For example, the i-th RNA-protein pair is positive example data, the corresponding label value is 1. A loss function can be calculated according to the interaction prediction value p(1|rᵢ,pᵢ), p(0|rᵢ, pᵢ) and the label value 1 of the RNA-protein pair to obtain a corresponding loss value. In the training process of the model, the interaction prediction value needs to be infinitely close to the label value, that is, to minimize the objective function. In an example, when the objective function needs to be minimized, a cross entropy loss function may be selected as the objective function. When the cross entropy loss function is calculated, if the label value is 1, the closer the p(1|rᵢ, pᵢ) is to 1, the smaller the calculated loss value is, and the closer the p(1|rᵢ, pᵢ), is to 0, the greater the calculated loss value is. Meanwhile, the closer the p(0|rᵢ, pᵢ) is to 1, the greater the calculated loss value is, and the closer the p(0|rᵢ, pᵢ) is to 0, the smaller the calculated loss value is. It may be understood that the cross entropy loss function is a performance function in the prediction model, and may be used to estimate the inconsistency degree between the prediction value of the prediction model and the label value. The smaller the calculated value of the cross entropy loss function is, the better the prediction effect of the model is.

Specifically, the cross entropy loss function may be:

loss=−Σᵢ₌₁^{K}(yᵢlogp(1|rᵢ, pᵢ)^{y}^{ᵢ}+(1−yᵢ)logp(0|rᵢ, pᵢ)^{(1-y}^{ᵢ}⁾) (20),

where rᵢ represents the i-th RNA sequence in the training data set, pᵢ represents the i-th protein sequence in the training data set, yᵢ represents the label value of the i-th RNA-protein pair in the training data set, p(1|rᵢ, pᵢ) represents the prediction value of presence of interaction in the i-th RNA-protein pair in the training data set, p(0|rᵢ, pᵢ) represents the prediction value of absence of interaction in the i-th RNA-protein pair in the training data set, and K is the total number of RNA-protein pairs in the training data set.

In step S1040, model parameters of the recurrent neural network and the selective attention mechanism model are adjusted according to the loss value.

Among them, the model parameter may be a weight parameter, a bias parameter, a parameter matrix, i.e., each mapping matrix W₁, W₂ and W₃ etc. For example, the model parameters of each model may be iteratively updated based on the calculated loss value, and when an iteration termination condition is satisfied, model parameter training of the plurality of interaction prediction models is completed. For example, the model parameters may be updated by using a stochastic gradient descent algorithm. According to the back propagation principle, an objective function such as a cross entropy loss function is continuously calculated, and model parameters of each model are updated at the same time according to the calculated loss value. when the objective function converges to the minimum value, training of all model parameters is completed. The model parameters may also be updated in a reverse iteration manner, and when a preset number of iterations is satisfied, training of all model parameters is completed. After the iteration is completed, the optimized model parameters may be obtained. In other examples, minimization objective functions such as the least squares method, the Adam optimization algorithm may also be alternately performed, and the model parameters may be sequentially updated from back to front to optimize the parameters.

In the above training process, parameters in the recurrent neural network and the selective attention mechanism model may be trained at the same time. For example, L is used as the objective function, and the mapping matrix W₃ in the full connection layer can be adjusted firstly. Since feature extraction needs to be performed on the first RNA vector sequence and the first protein vector sequence by using the selective attention mechanism model before binary classification prediction, and the RNA sequence to be predicted and the protein sequence need to be encoded by using the recurrent neural network, it can be further backpropagated to the selective attention mechanism model and the recurrent neural network, and model parameters in the selective attention mechanism model and the recurrent neural network and mapping matrices W₁ and W₂ are adjusted. Through a plurality of back propagation layer by layer, each model parameter tends to converge, or the training is terminated after a certain number of iterations is satisfied. Through this training method, the recurrent neural network and the selective attention mechanism model can be trained at the same time, so that the precision and accuracy of each model are ensured to be higher, and meanwhile, the training efficiency can be improved. After the training is completed, the probability value of interaction between the RNA sequence to be predicted and the protein sequence to be predicted can be predicted by using the finally obtained various models.

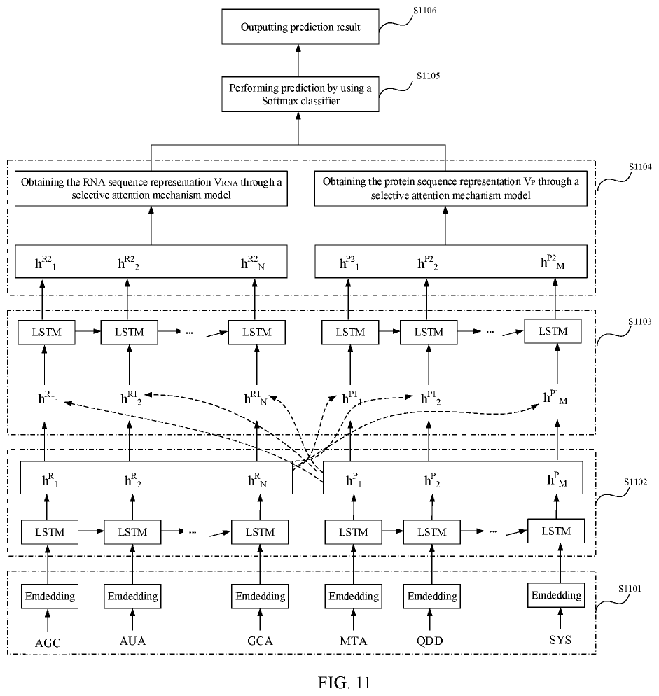

In a specific example embodiment, referring to FIG. 11, the probability value of interaction between the RNA sequence to be predicted and the protein sequence to be predicted may be predicted by using the trained attention mechanism model, the LSTM network, and the Softmax classifier according to steps S1101 to S1106.

In step S1101, the RNA sequence AGCAUA . . . GCA to be predicted is converted into N base 3-MER subsequences such as AGC, AUA, etc. Embedding encoding can be performed on each base 3-MER subsequence to obtain N base 3-mer Embedding vectors. The protein sequence MTAQDD . . . SYS to be predicted is converted into M amino acid 3-MER subsequences such as MTA, QDD, etc. Embedding encoding can be performed on each amino acid 3-mer subsequence to obtain M amino acid 3-mer Embedding vectors.

In step S1102, the obtained N base 3-mer Embedding vectors and M amino acid 3-mer Embedding vectors are respectively input into an LSTM network, and a vector hᵢ^{R} corresponding to each base 3-mer and a vector hᵢ^{P} corresponding to each amino acid 3-mer are output. The vectors hᵢ^{R} corresponding to N base 3-mer compose a first RNA vector sequence {hᵢ^{R}, i=1,2, . . . , N}, and the vectors hr corresponding to M amino acid 3-mer compose a first protein vector sequence {hⱼ^{P}, j=1,2, . . . , M}.

In step S1103, a relevance vector sequence yr of the first RNA vector sequence and a relevance vector sequence vⱼ^{R} of the first protein vector sequence are obtained through a selective attention mechanism model. A sequence {hᵢ^{R1}, i=1,2, . . . , N} is obtained by fusing the relevance vector sequence vᵢ^{P} of the first RNA vector sequence with the first RNA vector sequence, and a sequence {hⱼ^{P1}, j=1,2, . . . , M} is obtained by fusing the relevance vector sequence vⱼ^{R} of the first protein vector sequence with the first protein vector sequence. Then, the sequence {hᵢ^{R1}, i=1,2, . . . , N} is input into a LSTM network to capture the relevance relationship between the various base k-mer vectors in the sequence, and the sequence {hᵢ^{R2}, i=1,2, . . . , N} is output. In the same way, the sequence {hⱼ^{P1}, j=1,2, . . . , M} is input into the LSTM network to capture the relevance relationship between the various amino acid k-mer vectors in the sequence, and the sequence {hⱼ^{P2}, j=1,2, . . . , M} is output.

In step S1104, a self-relevance vector sequence {hᵢ^{R3}, i=1,2, . . . , N} of the sequence {hᵢ^{R2}, i=1,2, . . . , N} is obtained through a selective attention mechanism model, and average operation is performed on the self-relevance vector sequence to obtain a final representation v_{RNA} of the RNA sequence to be predicted. A self-relevance vector sequence {hⱼ^{P3}, j=1,2, . . . , M} of the sequence {hⱼ^{P2}, j=1,2, . . . , M} is obtained through a selective attention mechanism model, and average operation is performed on the self-relevance vector sequence to obtain a final representation v_{P} of the protein sequence to be predicted. The v_{RNA} and v_{P} are spliced to obtain a feature vector v to be predicted for interaction prediction.

In step S1105, binary classification prediction is performed on the feature vector v to be predicted by using a Softmax classifier to obtain an interaction prediction value between the RNA sequence to be predicted and the protein sequence to be predicted.

In step S1106, the interaction prediction value between the RNA sequence to be predicted and the protein sequence to be predicted is output to the terminal device for the user to view.

In an example embodiment of the present disclosure, at least one RNA sequence can also be obtained, and a protein sequence interacting with each input RNA sequence can be searched in a database. For example, after the user inputs at least one RNA sequence, each input RNA sequence may be combined with all protein sequences in the database into several RNA-protein pairs. Furthermore, interaction prediction may be performed on each RNA-protein pair according to steps S220 to S250. Specifically, a corresponding first RNA vector sequence and a corresponding first protein vector sequence can be obtained by encoding the RNA sequence and the protein sequence in each RNA-protein pair using a recurrent neural network. A relevance vector sequence of the first RNA vector sequence and a relevance vector sequence of the first protein vector sequence are obtained by using a selective attention mechanism model, and a second RNA vector sequence and a second protein vector sequence are obtained according to the relevance vector sequence of the first RNA vector sequence and the relevance vector sequence of the first protein vector sequence. A feature vector to be predicted is obtained by splicing the second RNA vector sequence and the second protein vector sequence. Finally, an interaction prediction value of each RNA-protein pair is obtained by using a classifier to perform classification prediction on the feature vector to be predicted. Among them, the interaction prediction value being 1 indicates that there is an interaction in the RNA-protein pair, and the interaction prediction value being 0 indicates that there is no interaction the RNA-protein pair. Then, all RNA-protein pairs with an interaction prediction value of 1 may be screened out, and the protein sequence in each RNA-protein pair is output to a terminal device for a user to view the protein sequence interacting with the input RNA sequence.

Similarly, in an example embodiment of the present disclosure, at least one protein sequence may also be obtained, and an RNA sequence interacting with each input protein sequence may be searched in a database. For example, after the user inputs at least one protein sequence, each protein sequence input may be combined with all RNA sequences in the database into several RNA-protein pairs. Furthermore, interaction prediction may be performed on each RNA-protein pair according to steps S 220 to 5250. Specifically, a corresponding first RNA vector sequence and a corresponding first protein vector sequence may be obtained by encoding the RNA sequence and the protein sequence in each RNA-protein pair. A relevance vector sequence of the first RNA vector sequence and a relevance vector sequence of the first protein vector sequence are obtained by using a selective attention mechanism model, and a second RNA vector sequence and a second protein vector sequence are obtained according to the relevance vector sequence of the first RNA vector sequence and the relevance vector sequence of the first protein vector sequence. A feature vector to be predicted is obtained by splicing the second RNA vector sequence and the second protein vector sequence. Finally, an interaction prediction value of each RNA-protein pair is obtained by using a classifier to perform classification prediction on the feature vector to be predicted. Among them, the interaction prediction value being 1 indicates that there is an interaction in the RNA-protein pair, and the interaction prediction value being 0 indicates that there is no interaction the RNA-protein pair. Then, all RNA-protein pairs with an interaction prediction value of 1 may be screened out, and the RNA sequence in each RNA-protein pair is output to a terminal device for a user to view the RNA sequence interacting with the input protein sequence.

In the method for RNA-protein interaction prediction provided by an example embodiment of the present disclosure, an RNA sequence to be predicted and a protein sequence to be predicted are obtained; a first RNA vector sequence is obtained by encoding the RNA sequence to be predicted; a first protein vector sequence is obtained by encoding the protein sequence to be predicted; a relevance vector sequence of the first RNA vector sequence and a relevance vector sequence of the first protein vector sequence are obtained through a selective attention mechanism model; and, the probability value of interaction between the RNA sequence to be predicted and the protein sequence to be predicted is determined according to the relevance vector sequence of the first RNA vector sequence and the relevance vector sequence of the first protein vector sequence. According to the present disclosure, the selective attention mechanism model is used to determine the relevance information between the RNA sequence and the protein sequence; and, the RNA sequence, the protein sequence and the relevance information can be fused. When the sequence information obtained by fusion is introduced to predict the probability value of interaction between the RNA sequence and the protein sequence, the accuracy of predicting the probability value of interaction between the RNA and the protein can be improved.

It should be noted that although the various steps of the methods in the present disclosure are described in a particular order in the drawings, this does not require or imply that these steps must be performed in that particular order, or that all of the illustrated steps must be performed to achieve the desired results. Additionally or alternatively, some steps may be omitted, a plurality of steps may be combined into one step for execution, and/or one step may be decomposed into a plurality steps for execution, etc.

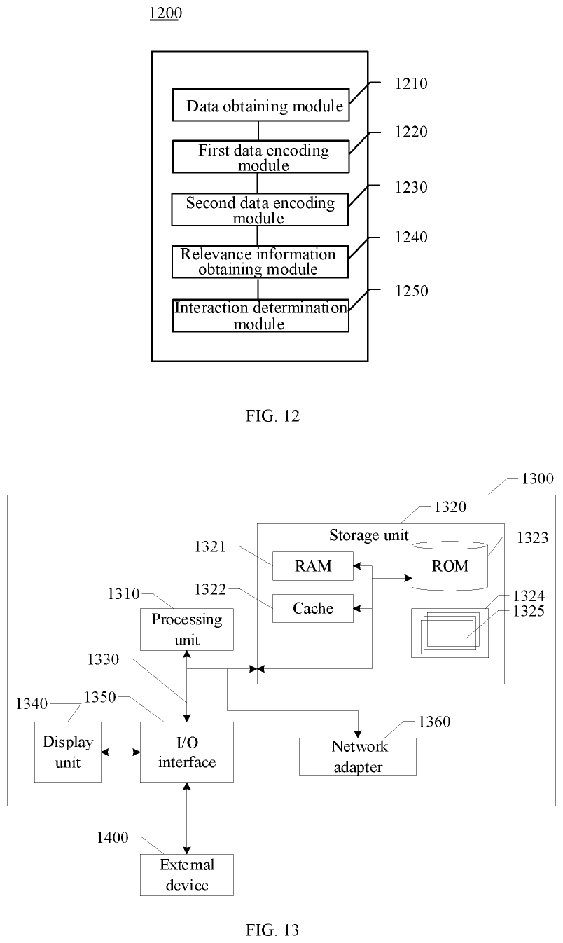

Furthermore, in this example embodiment, an apparatus for RNA-protein interaction prediction is further provided. The apparatus may be applied to a server or a terminal device. Referring to FIG. 12, the apparatus 1200 for RNA-protein interaction prediction may include a data obtaining module 1210, a first data encoding module 1220, a second data encoding module 1230, a relevance information obtaining module 1240, and an interaction determination module 1250.

The data obtaining module 1210 is configured to obtain an RNA-protein pair to be predicted.

The first data encoding module 1220 is configured to obtain a first RNA vector sequence by encoding the RNA sequence to be predicted.

The second data encoding module 1230 is configured to obtain a first protein vector sequence by encoding the protein sequence to be predicted.

The relevance information obtaining module 1240 is configured to obtain a relevance vector sequence of the first RNA vector sequence and a relevance vector sequence of the first protein vector sequence through a selective attention mechanism model.

The interaction determination module 1250 is configured to determine a probability value of interaction between the RNA sequence to be predicted and the protein sequence to be predicted according to the relevance vector sequence of the first RNA vector sequence and the relevance vector sequence of the first protein vector sequence.

In an optional embodiment, the first data encoding module 1220 includes:
a first sequence conversion module, configured to convert the RNA sequence to be predicted into N base k-mer subsequences;
a first sequence encoding module, configured to obtain the first RNA vector sequence by vectorizing each base k-mer subsequence.

In an optional embodiment, the first sequence encoding module includes:
a first sequence encoding unit, configured to obtain first vectors of N base k-mer subsequences by encoding each base k-mer subsequence;
a first vector operation unit, configured to obtain second vectors of N base k-mer subsequences by performing an operation on the first vectors of the N base k-mer subsequences using a first mapping matrix perform;
a first vector sequence determination unit, configured to compose the first RNA vector sequence with N base k-mer vectors by inputting the second vectors of the N base k-mer subsequences into a pre-trained recurrent neural network and outputting the N base k-mer vectors.

In an optional embodiment, the second data encoding module 1230 includes:
a second sequence conversion module, configured to convert the protein sequence to be predicted into M amino acid k-mer subsequences;
a second sequence encoding module, configured to obtain the first protein vector sequence by vectorizing each amino acid k-mer subsequence.

In an optional embodiment, the second sequence encoding module includes:
a second sequence encoding unit, configured to obtain first vectors of M amino acid k-mer subsequences by encoding each of the amino acid k-mer subsequences;
a second vector operation unit, configured to obtain second vectors of M amino acid k-mer subsequences by performing an operation on the first vectors of the M amino acid k-mer subsequences using a second mapping matrix;
a second vector sequence determination unit, configured to compose the first protein vector sequence with M amino acid k-mer vectors by inputting the second vectors of the M amino acid k-mer subsequences sequentially into a pre-trained recurrent neural network and outputting the M amino acid k-mer vectors.

In an optional embodiment, the relevance information obtaining module 1240 includes:

an RNA feature extraction unit, configured to obtain a first RNA hidden vector by performing feature extraction on the first RNA vector sequence;
a protein feature extraction unit, configured to obtain a first protein hidden vector by performing feature extraction on the first protein vector sequence;
a relevance information obtaining unit, configured to obtain the relevance vector sequence of the first RNA vector sequence and the relevance vector sequence of the first protein vector sequence by performing an operation on the first RNA hidden vector and the first protein hidden vector.

In an optional embodiment, the first RNA hidden vector includes a first RNA vector, a second RNA vector, and a third RNA vector. The RNA feature extraction unit is configured to: obtain the first RNA vector by performing an operation on the first RNA vector sequence using a first query weight matrix, obtain the second RNA vector by performing an operation on the first RNA vector sequence using a first key weight matrix, and obtain the third RNA vector by performing an operation on the first RNA vector sequence using a first value weight matrix.

In an optional embodiment, the first protein hidden vector includes a first protein vector, a second protein vector, and a third protein vector. the protein feature extraction unit is used for performing an operation on the first protein vector sequence by using a second query weight matrix to obtain the first protein vector; performing an operation on the first protein vector sequence by using a second key weight matrix to obtain the second protein vector; and performing an operation on the first protein vector sequence by using a second value weight matrix to obtain the third protein vector.

In an optional embodiment, the relevance information obtaining unit is configured to: obtain a first RNA attention score by calculating a similarity between the first RNA vector and the second protein vector, obtain the relevance vector sequence of the first RNA vector sequence by summing the third protein vector according to the first RNA attention score, obtain a first protein attention score by calculating a similarity between the first protein vector and the second RNA vector, and obtain the relevance vector sequence of the first protein vector sequence by summing the third RNA vector according to the first protein attention score.

In an optional embodiment, the interaction determination module 1250 includes:
a vector sequence determination unit, configured to obtain a second RNA vector sequence and a second protein vector sequence according to the relevance vector sequence of the first RNA vector sequence and the relevance vector sequence of the first protein vector sequence;
an interaction determination unit, configured to determine the probability value of interaction between the RNA sequence to be predicted and the protein sequence to be predicted according to the second RNA vector sequence and the second protein vector sequence.

In an optional embodiment, the vector sequence determination unit includes:
a first sequence splicing unit, configured to obtain an RNA fusion vector sequence by splicing the relevance vector sequence of the first RNA vector sequence and the first RNA vector sequence;
a first RNA vector sequence determination unit, configured to output the second RNA vector sequence by inputting the RNA fusion vector sequence into a pre-trained recurrent neural network;
a second sequence splicing unit, configured to obtain a protein fusion vector sequence by splicing the relevance vector sequence of the first protein vector sequence and the first protein vector sequence;
a first protein vector sequence determination unit, configured to output the second protein vector sequence by inputting the protein fusion vector sequence into a pre-trained recurrent neural network.

In an optional embodiment, the vector sequence determination unit includes:
a first sequence splicing unit, configured to obtain an RNA fusion vector sequence by splicing the relevance vector sequence of the first RNA vector sequence and the first RNA vector sequence;
a second sequence splicing unit, configured to obtain a protein fusion vector sequence by splicing the relevance vector sequence of the first protein vector sequence and the first protein vector sequence;
a second RNA vector sequence determination unit, configured to by obtain a self-relevance vector sequence of the RNA fusion vector sequence through a selective attention mechanism model and obtain the second RNA vector sequence according to the self-relevance vector sequence of the RNA fusion vector sequence;
a second protein vector sequence determination unit, configured to obtain a self-relevance vector sequence of the protein fusion vector sequence through a selective attention mechanism model and obtain the second protein vector sequence according to the self-relevance vector sequence of the protein fusion vector sequence.

In an optional embodiment, the second RNA vector sequence determination unit includes:
an RNA feature extraction unit, configured to obtain a second RNA hidden vector by performing feature extraction on the RNA fusion vector sequence;
an RNA self-relevance information determination unit, configured to obtain the self-relevance vector sequence of the RNA fusion vector sequence by performing an operation on the second RNA hidden vector;
an RNA self-relevance information operation unit, configured to obtain the second RNA vector sequence by performing an operation on the self-relevance vector sequence of the RNA fusion vector sequence.

In an optional embodiment, the second RNA hidden vector includes a fourth RNA vector, a fifth RNA vector, and a sixth RNA vector; the RNA feature extraction unit is configured to: obtain the fourth RNA vector by performing an operation on the RNA fusion vector sequence using a third query weight matrix; obtain the fifth RNA vector by performing an operation on the RNA fusion vector sequence using a third key weight matrix; and obtain the sixth RNA vector by performing an operation on the RNA fusion vector sequence using a third value weight matrix.

In an optional embodiment, the RNA self-relevance information determination unit is configured to: obtain an Second RNA attention score by calculating a similarity between the fourth RNA vector and the fifth RNA vector; and obtain the self-relevance vector sequence of the RNA fusion vector sequence by summing the sixth RNA vector according to the Second RNA attention score.

In an optional embodiment, the second protein vector sequence determination unit includes:
a protein feature extraction unit, configured to obtain a second protein hidden vector by performing feature extraction on the protein fusion vector sequence;
a protein self-relevance information determination unit, configured to obtain the self-relevance vector sequence of the protein fusion vector sequence by performing an operation on the second protein hidden vector;
a protein self-relevance information operation unit, configured to obtain the second protein vector sequence by performing an operation on the self-relevance vector sequence of the protein fusion vector sequence.

In an optional embodiment, the second protein hidden vector includes a fourth protein vector, a fifth protein vector, and a sixth protein vector; the protein feature extraction unit is configured to: obtain the fourth protein vector by performing an operation on the protein fusion vector sequence using a fourth query weight matrix; obtain the fifth protein vector by performing an operation on the protein fusion vector sequence using a fourth key weight matrix; and obtain the sixth protein vector by performing an operation on the protein fusion vector sequence using a fourth value weight matrix.

In an optional embodiment, the protein self-relevance information determination unit is configured to obtain a second protein attention score by calculating a similarity between the fourth protein vector and the fifth protein vector; and obtain the self-relevance vector sequence of the protein fusion vector sequence by summing the sixth protein vector according to the second protein attention score.

In an optional embodiment, the interaction determination module 1250 includes:
an original feature vector determination unit, configured to obtain a feature vector to be predicted by splicing the second RNA vector sequence and the second protein vector sequence;
a prediction value determination unit, configured to obtain an interaction prediction value between the RNA sequence to be predicted and the protein sequence to be predicted according to the feature vector to be predicted;
an interaction determination unit, configured to determine the probability value of interaction between the RNA sequence to be predicted and the protein sequence to be predicted according to the interaction prediction value.

In an optional embodiment, the interaction prediction value determination unit is configured to output a probability value of presence of interaction between the RNA sequence to be predicted and the protein sequence to be predicted by inputting the feature vector to be predicted into a classifier.

In an optional embodiment, the apparatus 1200 for RNA-protein interaction prediction further includes:
a training data obtaining module, configured to obtain a training data set, where the training data set includes a positive-example RNA-protein pair and a negative-example RNA-protein pair;
a prediction value output module, configured to determine an interaction prediction value of each RNA-protein pair in the training data set by using the recurrent neural network and the selective attention mechanism model;
a loss value calculation module, configured to obtain a corresponding loss value by calculating an interaction prediction value and a label value of each RNA-protein pair in the training data set using a loss function;
a model parameter adjustment module, configured to adjust model parameters of the recurrent neural network and the selective attention mechanism model according to the loss value.

In an optional embodiment, the apparatus 1200 for RNA-protein interaction prediction further includes:
a data output module, configured to output an interaction prediction result between the RNA sequence to be predicted and the protein sequence to be predicted.

The specific details of each module in the apparatus for RNA-protein interaction prediction are described in detail in the corresponding method for RNA-protein interaction prediction, and therefore, details are not described here again.

Each module in the above-mentioned apparatus may be a general-purpose processor, including: a central processor, a network processor, etc. It may also be a digital signal processor, an application specific integrated circuit, a field-programmable gate array or other programmable logic devices, a discrete gate or a transistor logic device, and a discrete hardware component. Each module may also be implemented in the form of software, firmware, etc. Each processor in the apparatus may be an independent processor, or may be integrated together.

An example embodiment of the present disclosure further provides a computer-readable storage medium storing with a program product capable of implementing the method described in the present description. In some possible embodiments, various aspects of the present disclosure may also be implemented in the form of a program product, including program code. When the program product is running on an electronic device, the program code is used to enable the electronic device to perform the steps of the various example embodiments of the present disclosure described in the above-mentioned “example method” part of the present description. The program product may employ a portable compact disk read-only memory (CD-ROM) and includes program code and may run on an electronic device, such as a personal computer. However, the program product of the present disclosure is not limited to this. In the present disclosure, the readable storage medium may be any tangible medium including or storing a program, and the program may be used by or combined with an instruction execution system, apparatus, or device.

The program product may employ any combination of one or more readable medium. The readable medium may be a readable signal medium or a readable storage medium. The readable storage medium may be, for example, but not limited to, an electrical, magnetic, optical, electromagnetic, infrared, or semiconductor system, apparatus, or device, or any combination of them. More specific examples of readable storage medium (non-exhaustive lists) include an electrical connection with one or more wires, a portable disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination of them.

The computer-readable signal medium may include a data signal that is propagated in a baseband or as part of a carrier, where readable program code is carried. Such a propagated data signal may take a variety of forms, including, but not limited to, an electromagnetic signal, an optical signal, or any suitable combination of them. The readable signal medium may also be any readable medium other than the readable storage medium. The readable medium may send, propagate, or transmit a program used by or combined with an instruction execution system, apparatus, or device.

The program code included on the readable medium may be transmitted by any suitable medium, including but not limited to wireless, wired, optical cable, RF, etc., or any suitable combination of the foregoing.

Program code for performing the operations of the present disclosure may be written in any combination of one or more programming languages. The programming languages include object-oriented programming languages, such as Java, C++, etc. The programming languages also includes conventional procedural programming languages, such as “C” languages or similar programming languages. The program code may be executed entirely on the user computing device, partly on the user device, as a stand-alone software package, partly on the user computing device and partly on the remote computing device, or entirely on the remote computing device or server. In situations involving a remote computing device, a remote computing device may be connected to a user computing device through any type of network, including a local area network (LAN) or a wide area network (WAN), or may be connected to an external computing device (e.g., being connected through the Internet via an Internet Service Provider).

An example embodiment of the present disclosure further provides an electronic device capable of implementing the above method. The electronic device 1300 according to the example embodiment of the present disclosure is described below with reference to FIG. 13. The electronic device 1300 shown in FIG. 13 is merely an example, and should not bring any limitation to the functions and usage scope of the embodiments of the present disclosure.

As shown in FIG. 13, the electronic device 1300 may be represented in the form of a general-purpose computing device. The components of the electronic device 1300 may include, but are not limited to, at least one processing unit 1310, at least one storage unit 1320, a bus 1330 connecting different system components (including the storage unit 1320 and the processing unit 1310), and a display unit 1340.

The storage unit 1320 stores a program code, and the program code may be executed by the processing unit 1310, so that the processing unit 1310 executes the steps according to various example embodiments of the present disclosure described in the foregoing “example method” part of the description. For example, the processing unit 1310 may execute any one or more of the method steps of FIG. 2 to FIG. 11.

The storage unit 1320 may include a readable medium in the form of a volatile storage unit, for example, a random access storage unit (RAM) 1321 and/or a cache storage unit 1322, and may further include a read-only storage unit (ROM) 1323.

The storage unit 1320 may also include a program/utility 1324 having a set of (at least one) program module 1325, including but not limited to: an operating system, one or more applications, other program modules and program data. Each of these examples or some combination of them may include an implementation of a network environment.

The bus 1330 may be one or more of several types of bus structures, including a storage unit bus or a storage unit controller, a peripheral bus, a graphics acceleration port, a processing unit, or a local bus using any of a plurality of bus structures.

The electronic device 1300 may also communicate with one or more external devices 1400 (e.g., a keyboard, a pointing device, a Bluetooth device, etc.), may also communicate with one or more devices that enable a user to interact with the electronic device 1300, and/or may communicate with any device (e.g., a router, a modem, etc.) that enables the electronic device 1300 to communicate with one or more other computing devices. Such communication may be performed through an input/output (I/O) interface 1350. Moreover, the electronic device 1300 may also communicate with one or more networks (e.g., a local area network (LAN), a wide area network (WAN), and/or a public network, such as the Internet) through the network adapter 1360. As shown, the network adapter 1360 communicates with other modules of the electronic device 1300 through the bus 1330. It should be understood that although not shown in the drawings, other hardware and/or software modules may be used in conjunction with the electronic device 1300, including but not limited to: microcode, device drivers, redundant processing units, external disk drive arrays, RAID systems, tape drives, and data backup storage systems, etc.

In some embodiments, the method for RNA-protein interaction prediction described in the present disclosure may be performed by the processing unit 1310 of the electronic device. In some embodiments, the RNA sequence to be predicted and the protein sequence to be predicted and the training data set for training each model may be input through the input interface 1350. For example, an RNA sequence to be predicted and a protein sequence to be predicted and a training data set for training each model are input through a user interaction interface of the electronic device. In some embodiments, the prediction result of the probability value of interaction between the RNA sequence to be predicted and the protein sequence to be predicted may be output to an external device 1400 through the output interface 1350 for a user to view.

Through the description of the above embodiments, those skilled in the art would easily understand that the example embodiments described here may be implemented by software, or may be implemented by software in combination with necessary hardware. Therefore, the technical solution according to the embodiments of the present disclosure may be embodied in the form of a software product. The software product may be stored in a non-volatile storage medium (which may be a CD-ROM, a USB flash disk, a mobile hard disk, etc.) or on a network, and may include several instructions to enable a computing device (which may be a personal computer, a server, a terminal device, or a network device, etc.) to perform a method according to an example embodiment of the present disclosure.

Furthermore, the above drawings are merely illustrative of the processes included in a method according to an example embodiment of the present disclosure, and are not intended to be limiting. It is easy to understand that the processes shown in the above drawings do not indicate or limit the temporal order of these processes. In addition, it is also easy to understand that these processes may be performed synchronously or asynchronously in a plurality of modules.

It should be noted that although several modules or units of a device for action execution are mentioned in the above detailed description, such partitioning is not mandatory. Indeed, according to embodiments of the present disclosure, the features and functions of two or more modules or units described above may be concretized within one module or unit. Conversely, the features and functions of one module or unit described above may be further divided into a plurality of modules or units to be concretized. It should be understood that the present disclosure is not limited to the precise structure that has been described above and illustrated in the accompanying drawings, and that various modifications and changes may be made without departing from the scope of the present disclosure. The scope of the present disclosure is limited only by the appended claims.

1. A method for RNA-protein interaction prediction, comprising:
obtaining an RNA sequence to be predicted and a protein sequence to be predicted;
obtaining a first RNA vector sequence by encoding the RNA sequence to be predicted;
obtaining a first protein vector sequence by encoding the protein sequence to be predicted;
obtaining a relevance vector sequence of the first RNA vector sequence and a relevance vector sequence of the first protein vector sequence through a selective attention mechanism model; and
determining a probability value of interaction between the RNA sequence to be predicted and the protein sequence to be predicted according to the relevance vector sequence of the first RNA vector sequence and the relevance vector sequence of the first protein vector sequence.

2. The method for RNA-protein interaction prediction according to claim 1, wherein obtaining a first RNA vector sequence by encoding the RNA sequence to be predicted comprises:
converting the RNA sequence to be predicted into N base k-mer subsequences; and
obtaining the first RNA vector sequence by vectorizing each base k-mer subsequence.

3. The method for RNA-protein interaction prediction according to claim 2, wherein obtaining the first RNA vector sequence by vectorizing each base k-mer subsequence comprises:
obtaining first vectors of N base k-mer subsequences by encoding each base k-mer subsequence;
obtaining second vectors of N base k-mer subsequences by performing an operation on the first vectors of the N base k-mer subsequences using a first mapping matrix;
composing the first RNA vector sequence with N base k-mer vectors by inputting the second vectors of the N base k-mer subsequences into a pre-trained recurrent neural network and outputting the N base k-mer vectors.

4. The method for RNA-protein interaction prediction according to claim 1, wherein obtaining a first protein vector sequence by encoding the protein sequence to be predicted comprises:
converting the protein sequence to be predicted into M amino acid k-mer subsequences; and
obtaining the first protein vector sequence by vectorizing each amino acid k-mer subsequence.

5. The method for RNA-protein interaction prediction according to claim 4, wherein obtaining the first protein vector sequence by vectorizing each amino acid k-mer subsequence comprises:
obtaining first vectors of M amino acid k-mer subsequences by encoding each amino acid k-mer subsequence;
obtaining second vectors of M amino acid k-mer subsequences by performing an operation on the first vectors of the M amino acid k-mer subsequences using a second mapping matrix; and
composing the first protein vector sequence with M amino acid k-mer vectors by inputting the second vectors of the M amino acid k-mer subsequences sequentially into a pre-trained recurrent neural network and outputting the M amino acid k-mer vectors.

6. The method for RNA-protein interaction prediction according to claim 1, wherein obtaining a relevance vector sequence of the first RNA vector sequence and a relevance vector sequence of the first protein vector sequence through a selective attention mechanism model comprises:
obtaining a first RNA hidden vector by performing feature extraction on the first RNA vector sequence;
obtaining a first protein hidden vector by performing feature extraction on the first protein vector sequence; and
obtaining the relevance vector sequence of the first RNA vector sequence and the relevance vector sequence of the first protein vector sequence by performing an operation on the first RNA hidden vector and the first protein hidden vector.

7. The method for RNA-protein interaction prediction according to claim 6, wherein the first RNA hidden vector comprises a first RNA vector, a second RNA vector, and a third RNA vector; and, obtaining a first RNA hidden vector by performing feature extraction on the first RNA vector sequence comprises:
obtaining the first RNA vector by performing an operation on the first RNA vector sequence using a first query weight matrix;
obtaining the second RNA vector by performing an operation on the first RNA vector sequence using a first key weight matrix; and
obtaining the third RNA vector by performing an operation on the first RNA vector sequence using a first value weight matrix.

8. The method for RNA-protein interaction prediction according to claim 7, wherein the first protein hidden vector comprises a first protein vector, a second protein vector, and a third protein vector; and, obtaining a first protein hidden vector by performing feature extraction on the first protein vector sequence comprises:
obtaining the first protein vector by performing an operation on the first protein vector sequence using a second query weight matrix;
obtaining the second protein vector by performing an operation on the first protein vector sequence using a second key weight matrix; and
obtaining the third protein vector by performing an operation on the first protein vector sequence using a second value weight matrix.

9. The method for RNA-protein interaction prediction according to claim 8, wherein obtaining the relevance vector sequence of the first RNA vector sequence and the relevance vector sequence of the first protein vector sequence by performing an operation on the first RNA hidden vector and the first protein hidden vector comprises:
obtaining a first RNA attention score by calculating a similarity between the first RNA vector and the second protein vector;
obtaining the relevance vector sequence of the first RNA vector sequence by summing the third protein vector according to the first RNA attention score;
obtaining a first protein attention score by calculating a similarity between the first protein vector and the second RNA vector; and
obtaining the relevance vector sequence of the first protein vector sequence by summing the third RNA vector according to the first protein attention score.

10. The method for RNA-protein interaction prediction according to claim 1, wherein determining a probability value of interaction between the RNA sequence to be predicted and the protein sequence to be predicted according to the relevance vector sequence of the first RNA vector sequence and the relevance vector sequence of the first protein vector sequence comprises:
obtaining a second RNA vector sequence and a second protein vector sequence according to the relevance vector sequence of the first RNA vector sequence and the relevance vector sequence of the first protein vector sequence; and
determining the probability value of interaction between the RNA sequence to be predicted and the protein sequence to be predicted according to the second RNA vector sequence and the second protein vector sequence.

11. The method for RNA-protein interaction prediction according to claim 10, wherein obtaining a second RNA vector sequence and a second protein vector sequence according to the relevance vector sequence of the first RNA vector sequence and the relevance vector sequence of the first protein vector sequence comprises:
obtaining an RNA fusion vector sequence by splicing the relevance vector sequence of the first RNA vector sequence and the first RNA vector sequence;
outputting the second RNA vector sequence by inputting the RNA fusion vector sequence into a pre-trained recurrent neural network;
obtaining a protein fusion vector sequence by splicing the relevance vector sequence of the first protein vector sequence and the first protein vector sequence; and
outputting the second protein vector sequence by inputting the protein fusion vector sequence into a pre-trained recurrent neural network.

12. The method for RNA-protein interaction prediction according to claim 10, wherein obtaining a second RNA vector sequence and a second protein vector sequence according to the relevance vector sequence of the first RNA vector sequence and the relevance vector sequence of the first protein vector sequence comprises:
obtaining an RNA fusion vector sequence by splicing the relevance vector sequence of the first RNA vector sequence and the first RNA vector sequence;
obtaining a protein fusion vector sequence by splicing the relevance vector sequence of the first protein vector sequence and the first protein vector sequence;
obtaining a self-relevance vector sequence of the RNA fusion vector sequence through a selective attention mechanism model and obtaining the second RNA vector sequence according to the self-relevance vector sequence of the RNA fusion vector sequence; and
obtaining a self-relevance vector sequence of the protein fusion vector sequence through a selective attention mechanism model and obtaining the second protein vector sequence according to the self-relevance vector sequence of the protein fusion vector sequence.

13. The method for RNA-protein interaction prediction according to claim 12, wherein obtaining a self-relevance vector sequence of the RNA fusion vector sequence through a selective attention mechanism model and obtaining the second RNA vector sequence according to the self-relevance vector sequence of the RNA fusion vector sequence comprises:
obtaining a second RNA hidden vector by performing feature extraction on the RNA fusion vector sequence;
obtaining the self-relevance vector sequence of the RNA fusion vector sequence by performing an operation on the second RNA hidden vector; and
obtaining the second RNA vector sequence by performing an operation on the self-relevance vector sequence of the RNA fusion vector sequence.

14. The method for RNA-protein interaction prediction according to claim 13, wherein the second RNA hidden vector includes a fourth RNA vector, a fifth RNA vector, and a sixth RNA vector;
obtaining a second RNA hidden vector by performing feature extraction on the RNA fusion vector sequence comprises:
obtaining the fourth RNA vector by performing an operation on the RNA fusion vector sequence using a third query weight matrix;
obtaining the fifth RNA vector by performing an operation on the RNA fusion vector sequence using a third key weight matrix; and
obtaining the sixth RNA vector by performing an operation on the RNA fusion vector sequence using a third value weight matrix;
obtaining the self-relevance vector sequence of the RNA fusion vector sequence by performing an operation on the second RNA hidden vector comprises:
obtaining a second RNA attention score by calculating a similarity between the fourth RNA vector and the fifth RNA vector; and
obtaining the self-relevance vector sequence of the RNA fusion vector sequence by summing the sixth RNA vector according to the second RNA attention score.

15. (canceled)

16. The method for RNA-protein interaction prediction according to claim 12, wherein obtaining a self-relevance vector sequence of the protein fusion vector sequence through a selective attention mechanism model and obtaining the second protein vector sequence according to the self-relevance vector sequence of the protein fusion vector sequence comprises:
obtaining a second protein hidden vector by performing feature extraction on the protein fusion vector sequence;
obtaining the self-relevance vector sequence of the protein fusion vector sequence by performing an operation on the second protein hidden vector; and
obtaining the second protein vector sequence by performing an operation on the self-relevance vector sequence of the protein fusion vector sequence.

17. The method for RNA-protein interaction prediction according to claim 16, wherein the second protein hidden vector comprises a fourth protein vector, a fifth protein vector, and a sixth protein vector;
obtaining a second protein hidden vector by performing feature extraction on the protein fusion vector sequence comprises:
obtaining the fourth protein vector by performing an operation on the protein fusion vector sequence using a fourth query weight matrix;
obtaining the fifth protein vector by performing an operation on the protein fusion vector sequence using a fourth key weight matrix; and
obtaining the sixth protein vector by performing an operation on the protein fusion vector sequence using a fourth value weight matrix;
obtaining the self-relevance vector sequence of the protein fusion vector sequence by performing an operation on the second protein hidden vector comprises:
obtaining a second protein attention score by calculating a similarity between the fourth protein vector and the fifth protein vector; and
obtaining the self-relevance vector sequence of the protein fusion vector sequence by summing the sixth protein vector according to the second protein attention score.

18. (canceled)

19. The method for RNA-protein interaction prediction according to claim 10, wherein determining the probability value of interaction between the RNA sequence to be predicted and the protein sequence to be predicted according to the second RNA vector sequence and the second protein vector sequence comprises:
obtaining a feature vector to be predicted by splicing the second RNA vector sequence and the second protein vector sequence;
obtaining an interaction prediction value between the RNA sequence to be predicted and the protein sequence to be predicted according to the feature vector to be predicted; and
determining the probability value of interaction between the RNA sequence to be predicted and the protein sequence to be predicted according to the interaction prediction value,
wherein obtaining an interaction prediction value between the RNA sequence to be predicted and the protein sequence to be predicted according to the feature vector to be predicted comprises:
outputting a probability value of presence of interaction between the RNA sequence to be predicted and the protein sequence to be predicted by inputting the feature vector to be predicted into a classifier.

20. (canceled)

21. The method for RNA-protein interaction prediction according to claim 1, further comprising:
obtaining a training data set, the training data set comprising a positive-example RNA-protein pair and a negative-example RNA-protein pair;
determining an interaction prediction value of each RNA-protein pair in the training data set by using a recurrent neural network and the selective attention mechanism model;
obtaining a corresponding loss value by calculating the interaction prediction value and a label value of each RNA-protein pair in the training data set using a loss function; and
adjusting model parameters of the recurrent neural network and the selective attention mechanism model according to the loss value.

22-23. (canceled)

24. A computer-readable storage medium, storing with a computer program thereon, wherein, when the computer program is executed by a processor, a method for RNA-protein interaction prediction is implemented, the method comprising:
obtaining an RNA sequence to be predicted and a protein sequence to be predicted;
obtaining a first RNA vector sequence by encoding the RNA sequence to be predicted;
obtaining a first protein vector sequence by encoding the protein sequence to be predicted;
obtaining a relevance vector sequence of the first RNA vector sequence and a relevance vector sequence of the first protein vector sequence through a selective attention mechanism model; and
determining a probability value of interaction between the RNA sequence to be predicted and the protein sequence to be predicted according to the relevance vector sequence of the first RNA vector sequence and the relevance vector sequence of the first protein vector sequence.

25. An electronic device, comprising:
a processor; and
a memory, configured to store an executable instruction of the processor;
wherein the processor is configured to execute a method for RNA-protein interaction prediction by executing the executable instruction, the method comprising:
obtaining an RNA sequence to be predicted and a protein sequence to be predicted;
obtaining a first RNA vector sequence by encoding the RNA sequence to be predicted;
obtaining a first protein vector sequence by encoding the protein sequence to be predicted;
obtaining a relevance vector sequence of the first RNA vector sequence and a relevance vector sequence of the first protein vector sequence through a selective attention mechanism model; and
determining a probability value of interaction between the RNA sequence to be predicted and the protein sequence to be predicted according to the relevance vector sequence of the first RNA vector sequence and the relevance vector sequence of the first protein vector sequence.
